(19) United States
(12) Patent Application Publication
(10) Pub. No.: US 2022/0111044 A1
(43) Pub. Date: Apr. 14, 2022

(54) WHOLE CELL TUMOR VACCINES AND METHODS OF USE THEROF

(71) Applicant: CHILDREN'S NATIONAL MEDICAL CENTER, Washington, DC (US)

(72) Inventors: Anthony SANDLER, Bethesda, MD (US); Xiaofang WU, Rockville, MD (US); Priya SRINIVASAN, Silver Spring, MD (US); Mousumi BASU, Rockville, MD (US)

(21) Appl. No.: 17/425,130

(22) PCT Filed: Jan. 29, 2020
(86) PCT No.: PCT/US20/15583

§ 371 Date: Jul. 22, 2021

Related U.S. Application Data
(60) Provisional application No. 62/798,258, filed on Jan. 29, 2019.

(51) Int. Cl. A61K 39/395; A61K 35/13; A61K 39/00; A61P 35/00; C07K 16/28; A61K 45/06

(52) U.S. Cl. CPC A61K 39/39558; A61K 35/13; A61K 39/0011; A61P 35/00; A61K 2039/507; C07K 16/2827; A61K 45/06; A61K 2039/575; A61K 2039/5152; C07K 16/2818

(57) ABSTRACT

Compositions and methods for the treatment of cancer are provided. Specifically, the disclosure provides a method for treating and/or inhibiting cancer or neoplasia in a subject, the method comprises contacting cancer cells obtained from the subject to be treated with an inhibitor of an immunity suppressing tumor protein; rendering the cancer cells proliferation-incompetent (e.g., by irradiation); and administering the treated cancer cells and a checkpoint inhibitor to the subject, wherein the inhibitor of an immunity suppressing tumor protein is an inhibitor of Inhibitor of differentiation protein 2 (Id2), Myc, and/or apolipoprotein E (ApoE).

This application claims priority under 35 U.S.C. § 119(e) to U.S. Provisional Patent Application No. 62/798,258, filed Jan. 29, 2019. The foregoing application is incorporated by reference herein.

### FIELD OF THE INVENTION

The present invention relates to immunotherapy. More specifically, novel whole cell tumor vaccines for treating cancer are provided.

### BACKGROUND OF THE INVENTION

Neuroblastoma is the most common extracranial solid tumor found in children and continues to have a poor prognosis in cases of high-risk disease, despite multimodal therapy (Brodeur, et al., Nat. Rev. Clin. Oncol. (2014) 11:704-713; Louis, et al., Annu. Rev. Med. (2015) 66:49-63; Maris, et al., Lancet (2007) 369:2106-2120). Immunotherapy in the form of either targeted antibodies or checkpoint inhibitors is changing cancer treatment, but many tumors are either nonimmunogenic or co-opt immunosuppressive pathways that evade immune-mediated clearance. Improved immunotherapies for treating cancers such as neuroblastoma are needed.

### SUMMARY OF THE INVENTION

In accordance with one aspect of the instant invention, methods for treating and/or inhibiting cancer or neoplasia in a subject are provided. In a particular embodiment, the method comprises contacting cancer cells obtained from the subject to be treated (e.g., autologous cells) with an inhibitor of an immunity suppressing tumor protein; rendering the cancer cells proliferation-incompetent (e.g., by irradiation); and administering the treated cancer cells and a checkpoint inhibitor to the subject. In a particular embodiment, the inhibitor of an immunity suppressing tumor protein is an inhibitor of Inhibitor of differentiation protein 2 (Id2), Myc, and/or apolipoprotein E (ApoE). In a particular embodiment, the inhibitor of an immunity suppressing tumor protein is an inhibitor of Myc. In a particular embodiment, the cancer cells are contacted/treated with JQ1 and/or I-BET726. In a particular embodiment, the checkpoint inhibitor is a programmed cell death (PD-1) inhibitor, programmed cell death-ligand 1 (PD-L1) inhibitor, and/or CTLA-4 inhibitor. In a particular embodiment, the checkpoint inhibitor is an antibody. In a particular embodiment, the subject is administered an anti-PD-L1 antibody and/or an anti-CTLA-4 antibody. The methods may further comprise administering an inhibitor of ApoE. The methods may further comprise administering an inhibitor of AP Endonuclease-1/Redox Effector Factor 1 (APE1/Ref-1) or contacting the cancer cells with an APE1/Ref-1 inhibitor, such as APC3330. The methods may further comprise obtaining a biological sample from the subject and/or isolating the cancer cells from a biological sample.

In accordance with one aspect of the instant invention, methods for stimulating an immune response to a tumor in a subject are provided. In a particular embodiment, the method comprises contacting cancer cells obtained from the tumor with an inhibitor of an immunity suppressing tumor protein; rendering the cancer cells proliferation-incompetent (e.g., by irradiation); and administering the treated cancer cells and a checkpoint inhibitor to the subject. In a particular embodiment, the inhibitor of an immunity suppressing tumor protein is an inhibitor of Inhibitor of differentiation protein 2 (Id2), Myc, and/or apolipoprotein E (ApoE). In a particular embodiment, the inhibitor of an immunity suppressing tumor protein is an inhibitor of Myc (e.g., 10058-F4). In a particular embodiment, the Myc inhibitor is an indirect inhibitor such as an inhibitor of Bromodomain and Extra-terminal motif (BET) proteins. Examples of BET inhibitors include JQ1 and/or I-BET726. In a particular embodiment, the cancer cells are contacted/treated with JQ1 and/or I-BET726. In a particular embodiment, the checkpoint inhibitor is a programmed cell death (PD-1) inhibitor, programmed cell death-ligand 1 (PD-L1) inhibitor, and/or CTLA-4 inhibitor. In a particular embodiment, the checkpoint inhibitor is an antibody. In a particular embodiment, the subject is administered an anti-PD-L1 antibody and/or an anti-CTLA-4 antibody. The methods may further comprise administering an inhibitor of ApoE. The methods may further comprise administering an inhibitor of AP Endonuclease-1/Redox Effector Factor 1 (APE1/Ref-1) or contacting the cancer cells with an APE1/Ref-1 inhibitor, such as APC3330. The methods may further comprise obtaining a biological sample from the subject and/or isolating the cancer cells from a biological sample.

In accordance with one aspect of the instant invention, compositions for performing the methods of the instant invention are also provided.

### BRIEF DESCRIPTION OF THE DRAWINGS

FIG. 1A shows PD-L1 expression on the surface of mouse Neuro2a (N2a) as analyzed by flow cytometry. PD-L1 expression is up-regulated in a dose-dependent manner (mean fluorescent intensity (MFT)). The expression of PD-L1 on human neuroblastoma cell lines SY5Y and SK-N-SH (non-NMYC amplified) had similar changes with exposure to IFNγ. FIG. 1B shows CD3 and PD-L1 expression in mouse neuroblastoma tumors following receipt of Id2kd vaccine with or without checkpoint blockade therapy. Representative tumors were dissected from naïve mice (I, V, IX, and XIII) and from mice after receipt of Id2kd vaccine (II, VI, X, and XIV), Id2kd plus PD-L1 antibody (III, VII, XI, and XV), and Id2kd plus CTLA-4 antibody (IV, VIII, XII, and XVI). Hematoxylin and eosin (H&E) staining (I±VIII) and immunofluorescence double staining (IX-XVI) for CD3 and PD-L1 were performed. The nuclei were stained with 4′,6-diamidino-2-phenylindole (DAPI). Representative micrographs from each cohort are shown. Areas of necrotic tissues are marked with black broken lines and coincide with areas of inflammatory cell infiltrates. Panel 1 (I-IV) and panel 3 (IX-XII) are 40× and 100× original magnification, respectively. The scale bar for (I-IV) is 500 μm and for (IX-XII) is 100 The enlarged images in panel 2 (V-VIII) and panel 4 (XIII-XVI) are of 600× original magnification. The scale bar is 20 FIG. 1C shows expression of activation markers on the surface of CD8+ tumor-infiltrating lymphocytes isolated from shrinking tumors of mice treated with α-CTLA-4 plus Id2kd vaccine. Programmed cell death 1 (PD1), TIM3, and LAG3 were expressed on tumor-infiltrating lymphocytes (TILs) by flow cytometry compared with isotype controls. APC: antigen-presenting cell; FITC, fluorescein isothiocyanate; IgG: immunoglobulin G; PerCP: peridinin-chlorophyll-protein complex.

FIG. 2A depicts the vaccination protocol and timeline. Briefly, A/J mice were inoculated with 1×10⁶ wild-type (WT) N2a, and once tumors were established, the mice were then vaccinated with various combinations of Id2kd-N2a, α-CTLA-4, and α-PD-L1 blocking antibodies. FIG. 2B shows tumor eradication in vaccinated mice (n=6) as detected by chemiluminescent imaging. FIG. 2C shows tumor growth in various treatment groups following vaccination. Ten out of 10 mice were cured of tumors when Id2kd-N2a vaccine was combined with inhibition of CTLA-4 and programmed cell death-ligand 1 (PD-L1) checkpoints. The graphs depict individual tumor growth over time and cure in parenthesis. FIG. 2D shows that average tumor growth (left panel) and survival (right) are markedly improved in the group receiving a combination of Id2kd N2a, α-CTLA-4, and α-PD-L1 when compared with other treatments (p=0.0007 for average tumor growth in untreated versus α-PD-L1+α-CTLA-4+Id2kd N2a, p=0.0005 for WT N2a+α-PD-L1 versus full combination, p=0.0025 for WT N2a+α-PD-L1+Id2kd N2a versus full combination, 2-way repeated measures ANOVA analysis, left panel; right panel p=0.0006 for survival trend, log-rank test; p=0.0007 in untreated control versus α-PD-L1+α-CTLA-4+Id2kd N2a, p=0.007 in α-PD-L1 versus combination, p=0.0008 in α-PD-L1+Id2kd N2a versus combination, p=0.0034 in α-PD-L1+α-CTLA-4 versus combination treatment, log-rank test).

FIGS. 3A and 3B show that PD-L1 blockade boosts interferon gamma (IFNγ) production of tumor-infiltrating lymphocytes (TILs) in vitro. CD8+ TILs isolated from tumors of mice treated with α-CTLA-4+ vaccine were cocultured with WT N2a cells at a 10:1 ratio, for 40 hours in an IFNγ Enzyme-Linked ImmunoSpot (ELISpot) assay. Where indicated, N2a cells were blocked with 10 μg/ml α-PD-L1 for 24 hours prior to coculture, and blocking was maintained during the assay. Also, as indicated, α-PD1 and α-TIM3 were added to the reactions at 10 μg/ml for the duration of the assay. FIG. 3A shows actual IFNγ spots/well imaged from ELISpot assay in duplicate. FIG. 3B graphs enumerated spots captured from an ELISpot reader in which each spot corresponds to a T cell producing IFNγ (unpaired 2-tailed Student t test, p<0.0485 were significant, p>0.0595 were not significant).

FIG. 4A shows a modified T-cell cytotoxicity assay using a caspase-3 cleavage assay. Interferon gamma (IFNγ) was used to up-regulate PD-L1 in wild-type (WT) cells. R1 represents the labeled tumor target cells, while R2 is the percentage of target cells positive for activated caspase-3. Drug cytotoxicity was induced by a combination of 1 μm staurosporine and 1 μm camptothecin, incubated for the same time as the other reactions. FIG. 4B shows long-term memory response of survivors. Splenocytes were isolated from mice at 6 months following cure with α-PD-L1+α-CTLA-4 plus vaccine, as well as from naïve mice. N2a cells were cocultured with splenocytes at a 1:10 ratio for 48 hours. Supernatants were tested for IFNγ expression levels by ELISA. Naïve controls produced no detectable IFNγ (Student t test, p=0.01; splenocyte IFNγ level with α-PD-L1 blockade).

FIGS. 5A and 5B show PD-L1 is expressed at 3.7-fold-lower levels on aggressive mouse neuroblastoma cell line AgN2a, as evidenced by flow cytometry (FIG. 5A) and real-time quantitative PCR (RT-qPCR) (p<0.005, Student t test; FIG. 5B). FIG. 5C shows exposure to even high levels of interferon gamma (IFNγ) does not up-regulate PD-L1 on AgN2a at 24 hours. FIG. 5D shows that the human NMYC-amplified IMR-32 cell line failed to up-regulate PD-L1 expression, unlike the other non-NMYC-amplified cell lines tested. FIG. 5E shows both CD3 and PD-L1 expression examined by immunofluorescence (IF) staining and confocal microscopy in WT 2a and AgN2a mouse tumors at baseline and following vaccination. Representative tumors were obtained from naïve mice and mice following receipt of Id2kd vaccine plus CTLA-4 antibody alone. The nuclei were stained with DAPI. Tissue sections were imaged at 200× original magnification, and the scale bar is 50 μm. IF staining demonstrates the minimal PD-L1 expression AgN2a tumors compared to WT N2a tumors, which explains the sensitivity of AgN2a to vaccine and anti-CTLA-4 alone, without anti-PD-L1 therapy.

FIG. 6A shows IF double staining of PD-L1 and CD3 performed on the paraffin-embedded neuroblastoma tumor tissue biopsied from high-risk (I-III), intermediate-risk (IV-VI), and low-risk (VII-IX) tumors. The nuclei were stained with DAPI, and the IF staining of CD3 was confirmed by immunohistochemical staining (X-XII). Tissue sections were imaged at 100× original magnification. The scale bar is 100 μm for (I-IX) and 50 μm for (X-XII). FIG. 6B shows the density of PD-L1 and CD3 staining as determined by digital image analysis shows significantly higher signal in low- and intermediate-risk groups compared to high-risk tumors. Each dot represents the mean fluorescent pixel area for a single subject. P-values were calculated with an unpaired Student t test.

FIG. 7A provides graphs of the expression of Myc expression as determined by real time PCR in B16 and N2a cells untreated (control) or treated with I-BET726 and JQ1 for 72 hours. Images of a Western blot analysis are also provided. FIG. 7B provides graphs of a flow cytometry analysis of untreated control cells or cells treated with BET (1 μm) or JQ1 (1 μm). Percentage of cells that were apoptotic, in S-phase, in G2-M phase, or in G0-G1 phase are indicated. FIGS. 7C and 7D provide graphs of quantitative RT-PCR analyses of gene expression in irradiated or un-irradiated N2a cells and B16 cells, respectively, following exposure to the Myc inhibitors (I-BET726 and JQ1) for 5 days. Statistically significant differences (*P<0.05, **P<0.01, ***P<0.001) between treated and untreated cohorts.

FIG. 8A provides a graph showing the suppression of Myc in B16 melanoma cells induced high levels of IFNγ secretion from pre-vaccinated splenocytes in co-culture. IFNγ was measured at 24 and 48 hours of culture (pg/ml). S: splenocytes; Treated: B16 cells expose to 1 μM BET and 1 μM JQ1 for 5 days; IRR: irradiated. FIG. 8B provides a graph of TNF in a co-culture with dendritic cells and untreated wild type B16 tumor cells or B16 tumor cells pre-treated with BET/JQ1. The triplicate of bars are, from left to right: unstimulated, stimulated with 0.1 μg/ml resiquimod, and stimulated with 0.5 μg/ml resiquimod.

FIG. 9A provides a schematic diagram of the therapeutic Myc targeted vaccine strategy plus checkpoint inhibitors and a tumor growth/survival curve in a neuroblastoma tumor models. Treated N2a in neuroblastoma model refers to Myc suppressed tumor cells. 6 of 8 mice (75%) were cured even in this non-MycN addicted cell line. FIG. 9B provides a schematic diagram of the therapeutic Myc targeted vaccine strategy plus checkpoint inhibitors and a tumor growth/survival curve in a melanoma tumor models. Historic control is an unvaccinated control, in which only wild type B16 were injected into the right leg. 75% of mice vaccinated (left leg) with Myc-targeted B16 treated cells combined with checkpoints and TLR agonist (n=4) survived at day 30, whereas all controls (n=5) or mice vaccinated with irradiated untreated cells plus checkpoints died from tumor burden (at day 13 and day 29, respectively).

FIGS. 10A and 10B show that ApoE suppresses T-cell function. The inhibition of Myc in B16 melanoma cells induced high levels of IFNγ secretion from naïve splenocytes in co-culture. IFNγ secretion was significantly suppressed in a dose dependent manner when exposure to ApoE agonist COG133 (0.3, 3 and 9 μM) (FIG. 10A); whereas blocking ApoE with an anti-ApoE antibody enhanced the production of IFNγ from activated splenocytes (FIG. 10B). IFNγ was measured at 48 hours of culture (pg/ml). S: splenocytes; NS: naïve splenocytes; VS: vaccinated splenocytes; S1S: splenocytes dissected from survivor mouse; Treated: B16 cells expose to 0.25 μM BET/0.25 μM JQ1 for 4 days.

### DETAILED DESCRIPTION OF THE INVENTION

The need for more effective therapy for tumors, including neuroblastoma and melanoma, is evident in the poor outcomes of high-risk or advanced disease. It is evident that immune based therapies—and specifically tumor vaccines—hold great promise. However, current immune based therapies are constrained by 1) antigen selection due to the high diversity of tumor antigens across patient tumors and 2) intrinsic tumor cell mechanisms enabling immune privilege/evasion. To circumvent both antigen selection and immune privilege/evasion, a personalized immunogenic whole cell vaccine is provided herein that carries patient specific antigens and overcomes immune privilege by targeting checkpoints and T-cell suppression to induce potent tumor immunity. Herein, it is shown that targeting Id2 protein or Myc in cancer cells (e.g., neuroblastoma or melanoma cells) creates an immunogenic whole cell vaccine which, when combined with checkpoint inhibitors, shrinks established tumors and cures mice of their disease. These findings demonstrate the power of whole cell vaccination and its ability to counter innate immune resistance.

In accordance with the instant invention, methods of treating and/or inhibiting a neoplasia or cancer in a subject are provided. The instant invention also encompasses methods of treating and/or preventing tumor progression or metastasis (including micrometastasis) in a subject having a neoplasia or cancer. The instant invention also encompasses methods of treating and/or reducing an established tumor (e.g., reversing the tumor load) in a subject. In a particular embodiment, the methods of the instant invention comprise contacting cancer cells obtained from the subject to be treated (e.g., autologous cells) with an inhibitor(s) of an immunity suppressing tumor protein; optionally rendering the cancer cells proliferation-incompetent; and administering a therapeutically effective amount of the treated cancer cells and, optionally, a checkpoint inhibitor(s), to the subject to be treated. The treated cancer cells and checkpoint inhibitor may be administered in the same composition (e.g., with a pharmaceutically acceptable carrier) or may be administered in separate compositions (the separate compositions may have the same or different pharmaceutically acceptable carriers). In a particular embodiment, the treated cancer cells are contacted with the checkpoint inhibitors prior to administration to the subject. The treated cancer cells and checkpoint inhibitor may be administered at the same time (e.g., simultaneously or concurrently) and/or at different times (e.g., consecutively (e.g., before and/or after)). In a particular embodiment, the method further comprises obtaining the cancer cells from a biological sample (e.g., tumor biopsy) from the subject to be treated and, optionally, culturing or growing the cancer cells (e.g., in vitro). The cancer cells may be cultured to increase the number of cells for manipulation and use in the methods of the instant invention. In a particular embodiment, the method further comprises obtaining the biological sample (e.g., tumor biopsy) from the subject to be treated.

Adaptive immune resistance induces an immunosuppressive tumor environment that enables a tumor immune evasion, thereby leading to tumor progression and escape. Tumors create the adaptive immune resistance through the expression of certain proteins which interact with the host's immune system. By inhibiting immunity suppressing tumor proteins, the instant invention has demonstrated that the immunosuppressive tumor environment can be reduced or eliminated. Immunity suppressing tumor proteins to be inhibited in the instant invention include, without limitation, Inhibitor of differentiation protein (e.g., Id1, Id2, Id3, and Id4; see, e.g., NCBI Gene ID: 3397, 3398, 3399, 3400), particularly at least Inhibitor of differentiation protein 2 (Id2; see, e.g., NCBI Gene ID: 3398); Myc (e.g., c-Myc and/or MycN; see, e.g., NCBI Gene ID: 4609 and 4613); and/or apolipoprotein E (ApoE; see, e.g., NCBI Gene ID: 348) or the receptor of ApoE (also known as Low density lipoprotein receptor-related protein 1 (LRP1); see, e.g., NCBI Gene ID: 4035). In a particular embodiment, the immunity suppressing tumor proteins to be inhibited is Id2 and/or Myc, particularly Myc (e.g., MycN and/or c-Myc). In a particular embodiment, inhibition of Myc results in the inhibition of MycN and c-Myc. In a particular embodiment, ApoE is inhibited in addition to Id2 and/or Myc. The inhibitors (e.g., antagonists) of the instant invention disrupt or suppress the function of the immunity suppressing tumor protein. In a particular embodiment, the inhibitor is an antibody or antigen-binding fragment thereof (e.g., an inhibitory antibody), an inhibitory nucleic acid molecule (e.g., an antisense, siRNA, or shRNA), or a small molecule inhibitor. In a particular embodiment, the inhibitor is an siRNA or shRNA. In a particular embodiment, the inhibitor is a small molecule. In a particular embodiment, the inhibitor is a direct inhibitor of Myc. For example, the Myc/Max interface may be targeted/inhibited to inhibit binding to DNA and the Myc transcriptional pathway. Examples of direct Myc inhibitors include, without limitation, 10058-F4 (5-[(4-ethylphenyl)methylene]-2-thioxo-4-thiazolidinone), KJ-Pyr-9 (4-[2-(2-Furanyl)-6-(4-nitrophenyl)-4-pyridinyl]benzamide), and omomyc (Beaulieu, et al. (2019) Sci. Translat. Med., 11(484):eaar5012). In a particular embodiment, the inhibitor is an inhibitor of BET (thereby leading to Myc suppression), particularly a small molecule inhibitor of BET. For example, an inhibitor of Bromodomain and Extra-terminal motif (BET) proteins (e.g., Bromodomain-containing protein 4 (BRD4)) can be used for indirect inhibition of Myc by blocking transcriptional initiation. Examples of small molecule BET inhibitors include, without limitation, JQ1 (tert-butyl 2-((6S)-4-(4-chlorophenyl)-2,3,9-trimethyl-6H-thieno[3,2-f][1,2,4]triazolo [4,3-a][1,4]diazepin-6-yl)acetate; e.g., (+)JQ1), I-BET (e.g., I-BET726 (GSK1324726A; Gosmini, et al., J. Med. Chem. (2014) 57(19):8111-8131); I-BET762 (molibresib, GSK525762); and I-BET151 (GSK1210151A)), dBET1 ((6S)-4-(4-chlorophenyl)-N-[4-[[2-[[2-(2,6-dioxo-3-piperidinyl)-2,3-dihydro-1,3-dioxo-1H-isoindol-4-yl]oxy]acetyl]amino]butyl]-2,3,9-trimethyl-6H-thieno[3,2-f][1,2,4]triazolo[4,3-a][1,4]diazepine-6-acetamide), and ARV-825 (2-((S)-4-(4-chlorophenyl)-2,3,9-trimethyl-6H-thieno[3,2-f][1,2,4]triazolo[4,3-a][1,4]diazepin-6-yl)-N-(4-(2-(2-(2-(2-((2-(2,6-dioxopiperidin-3-yl)-1,3-dioxoisoindolin-4-yl)amino)ethoxy)ethoxy)ethoxy)ethoxy)phenyl) acetamide). In a particular embodiment, the inhibitors JQ1 and/or I-BET (e.g., I-BET726) are used in the methods of the instant invention.

Checkpoint inhibitors target immune checkpoints which maintain self-tolerance and modulate immunity to prevent autoimmune side effects. In a particular embodiment, the immune checkpoint inhibitor is an antibody or antigen-binding fragment thereof (e.g., an inhibitory antibody), an inhibitory nucleic acid molecule (e.g., an antisense, siRNA, or shRNA), or a small molecule inhibitor. In a particular embodiment, the immune checkpoint inhibitor is an antibody or fragment thereof. Examples of immune checkpoint inhibitors include, without limitation: PD-1 inhibitors (e.g., antibodies, particularly monoclonal antibodies, immunologically specific for PD-1 such as pembrolizumab (Keytruda®) and nivolumab (Opdivo®)); programmed cell death-ligand 1 (PD-L1) inhibitors (e.g., antibodies, particularly monoclonal antibodies, immunologically specific for PD-L1 such as atezolizumab (Tecentriq®), avelumab (Bavencio®), and durvalumab (Imfinzi®); and CTLA-4 inhibitors (e.g., antibodies, particularly monoclonal antibodies, immunologically specific for CTLA-4 such as ipilimumab (Yervoy®)). In a particular embodiment, a CTLA-4 inhibitor and/or a PD-L1 or PD-1 inhibitor (particularly a PD-L1 inhibitor) are used in the methods of the instant invention (e.g., administered to the subject). In a particular embodiment, a PD-L1 and a CTLA-4 inhibitor are used in the methods of the instant invention (e.g., administered to the subject). In a particular embodiment, anti-PD-L1 antibodies and anti-CTLA-4 antibodies are used in the methods of the instant invention (e.g., administered to the subject).

In a particular embodiment, the method further comprises administering an inhibitor of AP Endonuclease-1/Redox Effector Factor 1 (APE1/Ref-1). In a particular embodiment, the APE1/Ref-1 inhibitor is an antibody or antigen-binding fragment thereof (e.g., an inhibitory antibody), an inhibitory nucleic acid molecule (e.g., an antisense, siRNA, or shRNA), or a small molecule inhibitor. In a particular embodiment, the APE1/Ref-1 inhibitor is a small molecule inhibitor. Examples of APE1/Ref-1 inhibitors include, without limitation, APX3330, APX2009 and APX2014 (Logsdon, et al., Sci. Rep. (2018) 8:13759). In a particular embodiment, the APE1/Ref-1 inhibitor is APX3330.

As explained hereinabove, the cancer cells may be rendered proliferation-incompetent prior to administration to the subject. Methods of rendering cells proliferation-incompetent include, without limitation, irradiation, freeze-thawing, exposing to chemotherapeutics, and high hydrostatic pressure. In a particular embodiment, the cancer cells are rendered proliferation-incompetent by irradiation. For example, the cancer cells may be irradiated for a sufficient enough amount of time (based on the intensity/strength of the radiation) to render the cancer cells proliferation incompetence.

In a particular embodiment, the methods may further compromise administering at least one additional cancer therapy to the subject. Additional cancer therapies include, without limitation, surgery (e.g., cryosurgery, laser surgery, resection), radiation therapy (e.g., external beam radiation, brachytherapy), hormone therapy, chemotherapy (e.g., administration of a chemotherapeutic agent), and chemoradiation therapy.

In a particular embodiment, the cancer that may be treated using the compositions and methods of the instant invention include, but are not limited to, prostate cancer, colorectal cancer, pancreatic cancer, cervical cancer, stomach cancer (gastric cancer), endometrial cancer, brain cancer, glioblastoma, neuroblastoma, liver cancer, bladder cancer, colon cancer, ovarian cancer, testicular cancer, vaginal cancer, uterine cancer, head and neck cancer, throat cancer, skin cancer, melanoma, basal carcinoma, mesothelioma, lymphoma, leukemia, esophageal cancer, breast cancer, rhabdomyosarcoma, sarcoma, lung cancer, small-cell lung carcinoma, non-small-cell carcinoma, adrenal cancer, thyroid cancer, renal cancer, bone cancer, and choriocarcinoma. In a particular embodiment, the cancer forms a tumor (e.g., a solid tumor). In a particular embodiment, the cancer is neuroblastoma.

In accordance with the instant invention, methods of producing an immune response, particularly a protective immune response, in a subject (e.g., a subject with cancer) are provided. The instant invention also encompasses methods of stimulating an immune response in a subject to a tumor. The instant invention also encompasses methods of inducing a neoplastic or cancer cell antigen-specific immune response in a subject. As described hereinabove, the methods of the instant invention comprise contacting cancer cells obtained from the subject to be treated (e.g., autologous cells) with an inhibitor(s) of an immunity suppressing tumor protein; optionally rendering the cancer cells proliferation-incompetent; and administering a therapeutically effective amount of the treated cancer cells and, optionally, a checkpoint inhibitor(s) to the subject to be treated. The treated cancer cells and checkpoint inhibitor may be administered in the same composition (e.g., with a pharmaceutically acceptable carrier) or may be administered in separate compositions (the separate compositions may have the same or different pharmaceutically acceptable carriers). In a particular embodiment, the treated cancer cells are contacted with the checkpoint inhibitors prior to administration to the subject. The treated cancer cells and checkpoint inhibitor may be administered at the same time (e.g., simultaneously or concurrently) and/or at different times (e.g., consecutively (e.g., before and/or after)). In a particular embodiment, the method further comprises obtaining the cancer cells from a biological sample (e.g., tumor biopsy) from the subject to be treated and, optionally, culturing or growing the cancer cells (e.g., in vitro). The cancer cells may be cultured to increase the number of cells for manipulation and use in the methods of the instant invention. In a particular embodiment, the method further comprises obtaining the biological sample (e.g., tumor biopsy) from the subject to be treated.

In accordance with the instant invention, methods of producing a whole cell tumor vaccine are provided. A “tumor vaccine” refers to a vaccine which, upon administration to a subject having a cancer or tumor, results in the reduction in tumor volume and/or cancer growth and/or increased survival of the subject. As described hereinabove, the methods of the instant invention comprise contacting cancer cells obtained from the subject to be treated (e.g., autologous cells) with an inhibitor(s) of an immunity suppressing tumor protein and, optionally, rendering the cancer cells proliferation-incompetent. The cells may be further contacted with a checkpoint inhibitor(s). The treated cancer cells may be contained with a composition comprising a pharmaceutically acceptable carrier. In a particular embodiment, the method further comprises obtaining the cancer cells from a biological sample (e.g., tumor biopsy) from the subject to be treated and, optionally, culturing or growing the cancer cells (e.g., in vitro). The cancer cells may be cultured to increase the number of cells for manipulation and use in the methods of the instant invention. In a particular embodiment, the method further comprises obtaining the biological sample (e.g., tumor biopsy) from the subject to be treated.

In accordance with another aspect of the instant invention, methods of treating and/or inhibiting a neoplasia or cancer in a subject are provided. The instant invention also encompasses methods of treating and/or preventing tumor progression or metastasis (including micrometastasis) in a subject having a neoplasia or cancer. The instant invention also encompasses methods of treating and/or reducing an established tumor (e.g., reversing the tumor load) in a subject. In a particular embodiment, the method comprises contacting cancer cells obtained from the subject to be treated (e.g., autologous cells) with a checkpoint inhibitor(s) and administering a therapeutically effective amount of the treated cancer cells and, optionally, the checkpoint inhibitor(s), to the subject to be treated. The method may further comprise rendering the cancer cells proliferation-incompetent. The treated cancer cells and checkpoint inhibitor may be administered in the same composition (e.g., with a pharmaceutically acceptable carrier) or may be administered in separate compositions (the separate compositions may have the same or different pharmaceutically acceptable carriers). In a particular embodiment, the treated cancer cells are contacted with the checkpoint inhibitors prior to administration to the subject. The treated cancer cells and checkpoint inhibitor may be administered at the same time (e.g., simultaneously or concurrently) and/or at different times (e.g., consecutively (e.g., before and/or after)). In a particular embodiment, the method further comprises obtaining the cancer cells from a biological sample (e.g., tumor biopsy) from the subject to be treated and, optionally, culturing or growing the cancer cells (e.g., in vitro). The cancer cells may be cultured to increase the number of cells for manipulation and use in the methods of the instant invention. In a particular embodiment, the method further comprises obtaining the biological sample (e.g., tumor biopsy) from the subject to be treated. In a particular embodiment, a CTLA-4 inhibitor and/or a PD-L1 or PD-1 inhibitor (particularly a PD-L1 inhibitor) are used in the methods. In a particular embodiment, a PD-L1 and a CTLA-4 inhibitor are used in the methods. In a particular embodiment, anti-PD-L1 antibodies and anti-CTLA-4 antibodies are used in the methods.

In accordance with another aspect of the instant invention, methods of producing a whole cell tumor vaccine are provided. In a particular embodiment, the method comprises contacting cancer cells obtained from the subject to be treated (e.g., autologous cells) with a checkpoint inhibitor(s). The method may further comprise rendering the cancer cells proliferation-incompetent. The treated cancer cells may be contained with a composition comprising a pharmaceutically acceptable carrier. In a particular embodiment, the method further comprises obtaining the cancer cells from a biological sample (e.g., tumor biopsy) from the subject to be treated and, optionally, culturing or growing the cancer cells (e.g., in vitro). The cancer cells may be cultured to increase the number of cells for manipulation and use in the methods of the instant invention. In a particular embodiment, the method further comprises obtaining the biological sample (e.g., tumor biopsy) from the subject to be treated. In a particular embodiment, a CTLA-4 inhibitor and/or a PD-L1 or PD-1 inhibitor (particularly a PD-L1 inhibitor) are used in the methods. In a particular embodiment, a PD-L1 and a CTLA-4 inhibitor are used in the methods. In a particular embodiment, anti-PD-L1 antibodies and anti-CTLA-4 antibodies are used in the methods.

In accordance with another aspect of the instant invention, compositions are provided comprising one or more of the above identified agents and a pharmaceutically acceptable carrier. In a particular embodiment, the composition comprises cancer cells (e.g., proliferation-incompetent cancer cells) comprising an inhibitor of an immunity suppressing tumor protein and a pharmaceutically acceptable carrier. The composition may further comprise a checkpoint inhibitor, as described above. In a particular embodiment, when the agents are contained in separate compositions as described above, the separate compositions are contained within a kit.

The compositions of the instant invention can be administered to an animal, particularly a mammal, more particularly a human, in order to treat, inhibit, or prevent the disease or disorder (e.g., cancer). As explained hereinabove, the compositions of the instant invention may also comprise at least one other therapeutic agent for treating, inhibiting, or preventing the disease or disorder (e.g., cancer). The additional therapeutic agent may also be administered in a separate composition. The compositions may be administered at the same time and/or at different times (e.g., sequentially).

The therapeutic agents described herein will generally be administered to a patient or subject as a pharmaceutical preparation. The term “patient” as used herein refers to human or animal subjects. These compositions may be employed therapeutically, under the guidance of a physician or other healthcare professional.

The compositions of the present invention can be administered by any suitable route, for example, by injection (e.g., for local (direct, including to or within a tumor) or systemic administration), oral, pulmonary, topical, nasal or other modes of administration. The composition may be administered by any suitable means, including subcutaneous, parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the compositions administered to the blood (e.g., intravenously), subcutaneously, or intraperitoneally. When more than one composition is administered, different routes of administration may be used for each composition.

In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (e.g., Remington: The Science and Practice of Pharmacy). The pharmaceutical composition of the present invention can be prepared, for example, in liquid form, or can be in dried powder form (e.g., lyophilized for later reconstitution).

The dose and dosage regimen of the therapeutic agents of the invention that is suitable for administration to a particular patient may be determined by a physician considering the patient's age, sex, weight, general medical condition, and the specific condition and severity thereof for which the therapeutic agent is being administered. The physician may also consider the route of administration, the pharmaceutical carrier, and the therapeutic agent's biological activity.

Selection of a suitable pharmaceutical preparation depends upon the method of administration chosen. For example, the therapeutic agents of the invention may be administered by direct injection into any cancerous tissue or into the area surrounding the cancer. In this instance, a pharmaceutical preparation comprises the therapeutic agents dispersed in a medium that is compatible with the cancerous tissue.

Therapeutic agents of the instant invention may also be administered parenterally by intravenous injection into the blood stream, or by subcutaneous, intramuscular, intrathecal, or intraperitoneal injection. Pharmaceutical preparations for parenteral injection are known in the art. If parenteral injection is selected as a method for administering the therapeutic agents, steps should be taken to ensure that sufficient amounts of the therapeutic agents reach their target cells to exert a biological effect.

Pharmaceutical compositions containing a therapeutic agent of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, topical, or parenteral. For parenterals, the carrier will usually comprise sterile water or saline, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment. The dosage units of the molecules may be determined individually or in combination with each anti-cancer therapy according to greater shrinkage and/or reduced growth rate of tumors.

The pharmaceutical preparation comprising the molecules of the instant invention may be administered at appropriate intervals until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of a disease or disorder herein may refer to curing, relieving, and/or preventing the disease or disorder, the symptom(s) of it, or the predisposition towards it.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule. The term “antibody” includes, but is not limited to, polyclonal antibodies, multiclonal antibodies, monoclonal antibodies, chimeric antibodies, humanized and primatized antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof; antibody fragments such as Fab fragments, F(ab′) fragments, single-chain FvFcs, single-chain Fvs; and derivatives thereof including Fc fusions and other modifications, and any other immunologically active molecule so long as they exhibit the desired biological activity (i.e., antigen association or binding). Moreover, the term further includes all classes of antibodies (i.e. IgA, IgD, IgE, IgG, and IgM) and all isotypes (i.e., IgG1, IgG2, IgG3, IgG4, IgA1, and IgA2), as well as variations thereof unless otherwise dictated by context.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, a “biological sample” refers to a sample of biological material obtained from a subject, particularly a human subject, including a tissue, a tissue sample, cell(s), and a biological fluid (e.g., blood, blood fraction, serum, or urine). A biological sample or tumor biopsy may be obtained in the form of, e.g., a tissue biopsy, such as, an aspiration biopsy, a brush biopsy, a surface biopsy, a needle biopsy, a punch biopsy, an excision biopsy, an open biopsy, an incision biopsy and an endoscopic biopsy. A tumor sample or biopsy may be obtained, for example, by the surgical removal of tissue from within a patient and/or tissue obtained from an excised organ or tissue or fluid.

Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide, nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; and tubulin interactive agents (e.g., vincristine, vinblastine, and paclitaxel (Taxol®)).

Radiation therapy refers to the use of high-energy radiation from x-rays, gamma rays, neutrons, protons and other sources to target cancer cells. Radiation may be administered externally or it may be administered using radioactive material given internally. Chemoradiation therapy combines chemotherapy and radiation therapy.

The phrase “small, interfering RNA (siRNA)” refers to a short (typically less than 30 nucleotides long, particularly 12-30 or 19-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al., Current Protocols in Molecular Biology, John Wiley and Sons, Inc). As used herein, the term siRNA may include short hairpin RNA molecules (shRNA). Typically, shRNA molecules consist of short complementary sequences separated by a small loop sequence wherein one of the sequences is complimentary to the gene target. shRNA molecules are typically processed into an siRNA within the cell by endonucleases. Exemplary modifications to siRNA molecules are provided in U.S. Application Publication No. 20050032733. Expression vectors for the expression of siRNA molecules may employ a strong promoter which may be constitutive or regulated. Such promoters are well known in the art and include, but are not limited to, RNA polymerase II promoters, the T7 RNA polymerase promoter, and the RNA polymerase III promoters U6 and Hl.

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods.

As used herein, the term “kit” refers to a delivery system (e.g., box or container) for delivering materials. The delivery system allows for the storage, transport, or delivery of materials/compositions and/or supporting materials (e.g., instructions for using the materials). A “kit” may include one or more enclosures (e.g., boxes or containers) containing the relevant materials/compositions and/or supporting materials. For example, when two or more separate enclosures are used, each enclosure contains a subportion of the total kit components.

The following examples are provided to illustrate various embodiments of the present invention. They are not intended to limit the invention in any way.

### Example 1

The immune system has inhibitory pathways that maintain self-tolerance and modulate immunity to prevent autoimmune side effects (Pardoll, D. M., Nat. Rev. Cancer (2012) 12:252-264). These inhibitory pathways, known as “checkpoints,” are also exploited by tumors to dampen and evade antitumor immunity. CTLA-4 is a key molecule expressed on the surface of T cells. It down-regulates the T cell's response when the immune system is activated. Hence, blocking its function, either alone or in combination with other therapies, leads to improved T-cell activation and expansion (Schwartz, R. H., Cell (1992) 71:1065-1068; Lenschow, et al., Annu. Rev. Immunol. (1996) 14:233-258; Egen, et al., Immunity (2002) 16:23-35). Programmed cell death 1 (PD1) is another immune checkpoint receptor and is more broadly expressed on T cells than CTLA-4 (Sfanos, et al., Prostate (2009) 69:1694-1703; Ahmadzadeh, et al., Blood (2009) 114:1537-1544). It is proposed to function downstream in the immune response, limiting the activity of T cells in peripheral tissues that express PD-L1, and thus reduce autoimmunity (Ishida, et al., EMBO J. (1992) 11:3887-3895; Freeman, et al., J. Exp. Med. (2000) 192:1027-1034; Keir et al., Annu. Rev. Immunol. (2008) 26:677-704). PD-L1 is expressed on the surface of many tumors as well, but the benefit of blocking the PD1/PD-L1 axis for immunotherapy has not been defined in neuroblastoma.

In order to induce effective immunity against a tumor, increased immunogenicity of the tumor itself is necessary. Id2 knockdown of mouse neuroblastoma (Id2kd-N2a) cells are rejected by most mice following inoculation and that the same mice then fail to grow tumors when subsequently rechallenged with wild-type Neuro2a cells. Antibody depletion of CD8+ cells or immune-incompetent mice grow Id2kd tumors avidly, validating the concept that Id2 knockdown confers tumor cell immunogenicity in immune-competent hosts. Thus, Id2kd tumor cells can be used as whole cell vaccines, in which the altered tumor cells themselves are administered back to the host as a vaccine to induce antitumor immunity. Acting in concert with a costimulatory CTLA-4 checkpoint inhibitor, Id2kd-N2a whole tumor cell vaccination generated a potent tumor-specific T-cell response, capable of eradicating established tumors in 60% of mice (Chakrabarti, et al., PLoS ONE (2013) 8:e83521; Chakrabarti, et al., PLoS ONE (2015) 10:e0129237). Surprisingly, in the same strain of mice, this vaccine approach was even more effective in a nonimmunogenic, aggressive (AgN2a) model, indicating a less immunosuppressive tumor microenvironment (Chakrabarti, et al., PLoS ONE (2015) 10:e0129237). When CTLA-4 was used alone without vaccination in the wild-type (WT) N2a model or the AgN2a model, only 40% and 0% of mice were cured of tumor, respectively (Chakrabarti, et al., PLoS ONE (2015) 10:e0129237).

Herein, the role of PD-L1 checkpoint inhibition in neuroblastoma is investigated. It is shown that PD-L1 is expressed on mouse and human neuroblastoma and is up-regulated following interferon gamma (IFNγ) treatment or T-cell tumor infiltration. CTLA-4 blockade plus Id2kd vaccination induces tumor specific T-cell expansion and tumor infiltration in mice, in which the infiltrating CD8 T cells are characterized by PD1 expression. The combination of Id2kd-N2a cell vaccination with anti CTLA-4 plus anti PD-L1 antibody treatment proved to be highly effective, even against established neuroblastoma tumors, resulting in cure of treated mice (n=16) as well as long-term immune memory (6 months). In a nonimmunogenic, aggressive neuroblastoma model (AgN2a), PD-L1 expression is neither significant nor up-regulated in response to IFNγ and T-cell infiltrates, making the tumor more susceptible to vaccine therapy. Characteristically, tumor infiltration of T cells and PD-L1 expression seem to also be associated with risk stratification in human neuroblastoma tumors. Low- and intermediate-risk tumors have abundant infiltrating T cells that are surrounded with high PD-L1 tumor expression, while high-risk tumors lack significant T-cell infiltrates and PD-L1 expression.

### Materials and Methods

### Animals

Female A/J mice aged 6 weeks were purchased from Jackson Laboratories (Bar Harbor, Me.). The animals were acclimated for 4-5 days prior to tumor challenge. All procedures were approved by the Institutional Animal Care and Use Committee (IACUC) of Children's National Medical Center, Washington, D.C.

### Cells

The murine neuroblastoma cell line Neuro2a (N2a) is derived from an A/J mouse and was purchased from the American Type Culture Collection (ATCC, Manassas, Va.). The aggressive N2a subclone AgN2a was derived from repeated in vivo passaging of these cells as described (Chakrabarti, et al., PLoS ONE (2015) 10:e0129237). The cells were maintained in Dulbecco's Modified Essential Medium (DMEM) supplemented with 1% penicillin-streptomycin (Invitrogen, Carlsbad, Calif.) and 10% fetal bovine serum (Gemini Bioproducts, Sacramento, Calif.). Mouse splenocytes were cultured in RPMI medium supplemented with 2 mM L-glutamine, 10% fetal bovine serum, and 1% penicillin-streptomycin. Cells were grown at 37° C. under 5% CO₂.

Human neuroblastoma cell lines IMR-32, SK-N-SH, and SH-SY5Y were obtained from the ATCC. IMR-32 and SK-N-SH cells were grown in ATTC Eagle's Minimum Essential Medium (EMEM) supplemented with 10% FBS. SH-SY5Y cells were cultured in ATCC EMEM mixed 1:1 with F12 medium, and FBS was added to a final concentration of 10%.

### Whole Tumor Cell Vaccine

Id2kd-N2a whole tumor cells were generated as described (Chakrabarti, et al., PLoS ONE (2015) 10:e0129237). The anchorage-dependent Neuro2a cells were transduced with Id2-shRNA expressing lentiviral particles containing a puromycin resistance gene (Santa Cruz Biotechnology, Santa Cruz, Calif.) for stable knockdown of Id2. The stable clones expressing the Id2-shRNA (Id2kd-N2a) were selected using puromycin according to the manufacturer's instructions.

### Specimens and Patient Demographics

Human specimens were obtained from 13 patients diagnosed with low-risk (n=3), intermediate-risk (n=5), and high-risk (n=5) neuroblastoma. Diagnosis and staging were performed according to Children's Oncology Group (COG) protocols. Biopsies were taken at the time of diagnosis and prior to initiation of any therapy. Specimen collection was obtained after appropriate research consents (and assents when applicable).

### Antibodies and Reagents

Anti (α)-mouse CTLA-4, α-mouse PD-L1, and mouse IgG2b isotype antibodies were purchased from BioXCell® (West Lebanon, N.H.). Mouse α-CD4 APC, α-CD8 PerCP Cy5.5, α-PD-L1, and purified α-mouse CD3 were bought from BD Biosciences (San Jose, Calif.). Mouse α-CD45 PE, α-PD1 FITC, α-TIM3 APC, and α-LAG3 APC were purchased from eBioscience and Biolegend (San Diego, Calif.). α-mouse and α-human recombinant IFNγ was purchased from Peprotech (Rocky Hill, N.J.).

### Mouse Neuroblastoma Therapy Models

A/J mice were injected subcutaneously (s.c.) in the right flank with 1×10⁶ freshly prepared tumor (Neuro2a or AgN2a or N2α-luc) cells in 100 μl phosphate-buffered saline (PBS) on day 0. One million Id2kd-N2a cells were injected (s.c.) into the left flank of each mouse on day 5 and again on day 12 as a whole cell vaccine. The mice usually developed tumors of 5 mm in size on the right flank by day 6. Anti-CTLA-4 and anti-PD-L1, each at a dose of 100 μg/mouse/time point, were administered intraperitoneally on days 5, 8, and 11. Mice were monitored daily following tumor inoculation. Tumor growth was recorded on alternate days by measuring the diameter in 2 dimensions using a caliper and by imaging the mice for tumor bioluminescence using IVIS Lumina III (Perkin Elmer, Houston, Tex.) when appropriate. Tumor volume was calculated using the following formula: (large diameter×small diameter)²×0.52. A tumor size of 20 mm in diameter in any dimension was designated as the endpoint, and mice were euthanized at that time. Euthanasia was achieved through cervical dislocation after CO₂ narcosis. If a tumor impaired the mobility of an animal, became ulcerated, or appeared infected, or a mouse displayed signs of “sick mouse posture,” the mouse was euthanized. Food was provided on the cage floor when the tumor size reached 15 mm in diameter. All the procedures are approved by the IACUC at CNMC and are in accordance with the humane care of research animals.

### Isolation of Tumor-Infiltrating Lymphocytes and Splenocytes

Mouse tumors were harvested, mechanically disrupted, and then digested with a cocktail of collagenase I, dispase II, and DNase 1 (Sigma Aldrich, MO) as per the method described (Chakrabarti, et al., PLoS ONE (2015) 10:e0129237). CD8+ T cells were isolated from the tumor digest by positive selection using the mouse CD8a+ T-cell isolation kit (Miltenyi Biotec, San Diego, Calif.). Spleens were collected from mice euthanized by CO₂ narcosis and cervical dislocation. Spleens were pulverized through a 40-μm mesh cell strainer and treated with ACK lysing buffer to remove erythrocytes before being cultured in RPMI medium.

### Characterization of Mouse Tumors by Immunofluorescence (IF)

Mouse tumors were excised either when they reached 10 mm or when they started to shrink following vaccine therapy. Specimens were fixed in 10% neutral buffered formalin (pH 6.8-7.2; Richard-Allan Scientific, Kalamazoo, Mich.) for paraffin embedding and sectioning. Five μm tissue sections were cut with a microtome, and sample processing and IF staining were performed as described using the following primary antibodies: CD3 rabbit anti-mouse mAb (1:100, ab16669, Abcam, Cambridge, Mass.) and PD-L1 goat anti-mouse polyclonal Ab (1:20, AF 1019, R&D Systems, Minneapolis, Minn.). Isotype-matched antibodies were used for negative controls. Sections were mounted with ProLong™ Diamond Antifade Mountant with DAPI (Thermo Fisher Scientific, Halethorpe, Md.).

### Flow Cytometry

Cells from mouse tumor digests and mouse splenocytes were stained with the fluorescently conjugated antibodies described above. Flow cytometry was done using a Becton Dickinson/Cytek FACSCalibur™ (BD Biosciences, San Jose, Calif.). Data were analyzed using the FlowJo program (Treestar, Ashland, Oreg.).

### IFNγ Measurement

A total of 2×10⁴ freshly isolated mouse splenocytes were plated in a volume of 200 μl per well of 96-well round bottom plates. Splenocytes were stimulated with 2×10⁵ WT N2a cells and 1.0 μg/ml α-CD3. WT N2a was blocked with 10 μg/ml α-mouse PD-L1, α-mouse CTLA-4 antibody, or IgG2b isotype control for 24 hours prior to interaction with splenocytes. Blocking was continued at the same concentration during the interaction with the splenocytes. Plates were incubated at 37° C. under 5% CO₂ for 48 hours. Supernatants were collected from triplicate wells, and IFNγ was assayed using the Ready-set-go mouse IFNγ ELISA kit from Ebioscience (San Diego, Calif.). Readings were measured at 450 nm using the EnSpire™ 2300 Multilabel plate reader (Perkin Elmer, Waltham, Mass.).

An Enzyme-Linked ImmunoSpot (ELISpot) assay was performed in duplicate under the same cell conditions listed above, using the mouse IFNγ ELISpot Basic kit (Mabtech, Cincinnati, Ohio). Counting of spots and data analysis were carried out by ZellNet Consulting (Fort Lee, N.J.).

### Cytotoxicity Assay

A modified flow-cytometry-based cytotoxicity assay detecting the presence of activated caspase 3 in target cells was performed. WT N2a target cells were labeled with CellTrace™ Far Red stain (Invitrogen, Carlsbad, Calif.). Tumor-infiltrating CD8+ T cells (CD8 TILs) were isolated from established tumors of A/J mice following complete vaccination with Id2Kd N2a, plus anti-CTLA-4+ anti-PD-L1. Effector cells to target cells were incubated at a 1:20 ratio for 3 hours at 37° C. The cells were then fixed and stained with PE-conjugated activated caspase 3 antibody (BD Biosciences, San Diego, Calif.). Target cells with no added effectors were used to determine spontaneous death. Drug-induced killing of tumor targets was determined by incubation with 1 μm campothecin and 1 μm staurosporine for 3 hours at 37° C. Gating and flow cytometry analysis was carried out according to the protocol described (He, et al., J. Immunol. Methods (2005) 304:43-59).

### Fluorescent Multiplex Immunohistochemistry

Five-micron-thick formalin-fixed paraffin-embedded (FFPE) human neuroblastoma tissue sections were deparaffinized in xylene and hydrated with graded alcohol and distilled water. Antigen retrieval was performed in EDTA unmasking solution (cell signaling) using a vegetable steamer for 15 minutes. This was followed by blocking of endogenous peroxidase activity with 3% hydrogen peroxide for 10 minutes (Sigma, Bellefonte, Pa.). After rinsing the slides in PBS, the slides were incubated with CD3c (D7A6E) XP Rabbit anti-human mAb (1:250, #85061, Cell signaling) for 1 hour at room temperature (RT). The antigen-antibody reaction was boosted by SignalStain® Boost Detection Reagent for 30 minutes. Following a wash, the slides were incubated with the Tyramide (TSA)-plus Cyanine 3 (NEL744001KT, PerkinElmer, Life Technologies) at 1:100 dilution for 10 minutes. For double staining with PD-L1, the slides were brought to a boil, the antibody-antigen reaction for CD3 was stripped in 10 mM sodium citrate buffer (PH=6, #14746, cell signaling) for 10 minutes, and then repeat staining/boosting/detection was performed using PD-L1 (E1L3N) XP Rabbit antihuman mAb (1:250, #77563, Cell signaling) and TSA-plus FITC (NEL741001KT, PerkinElmer, Life Technologies). Sections were then mounted with ProLong™ Diamond Antifade Mountant with DAPI (Thermo Fisher Scientific).

### Confocal Microscopy Imaging

IF-stained markers were observed with individual sections (xy plane). Confocal images were acquired with a Zeiss LSM 510 confocal microscope (Carl Zeiss MicroImaging, Thornwood, N.Y.) using Zen 2010 Light Edition acquisition software. Images were taken at magnifications of 100× and 630× under oil immersion.

### Quantitative Analysis of PD-L1 and CD3 Expression in Human Tissue

Ten to fifteen randomly selected fields in each stained specimen were imaged under 100× magnification. Quantification of fluorescent intensity was achieved using Olympus cellSens imaging software (version 1.7). The fluorescent intensity in each field was measured using a manual threshold setting. Measurements were made by the same person, and this individual was blind to the identity of the specimen. Data were presented as the mean fluorescent intensity of all the fields for each specimen.

### Statistical Analysis

The specific tests used to analyze each set of experiments are indicated in the figure legends. For each statistical analysis, appropriate tests were selected on the basis of whether the data was normally distributed by using the D'Agostino-Pearson normality test. Data were analyzed using an unpaired 2-tailed Student t test for comparisons between 2 groups and 2-way repeated-measures ANOVA to compare differences between average tumor growth curves. Survival curves were calculated according to the Kaplan-Meier method; survival analyses were performed using the log-rank test. Statistical calculations were performed using GraphPad Prism software (GraphPad Software, San Diego, Calif., US), and the probability level of p<0.05 was considered significant.

### Results

### PD-L1 is Expressed on Both Mouse and Human Neuroblastoma and is Upregulated by IFNγ Exposure or Tumor-Infiltrating T Cells

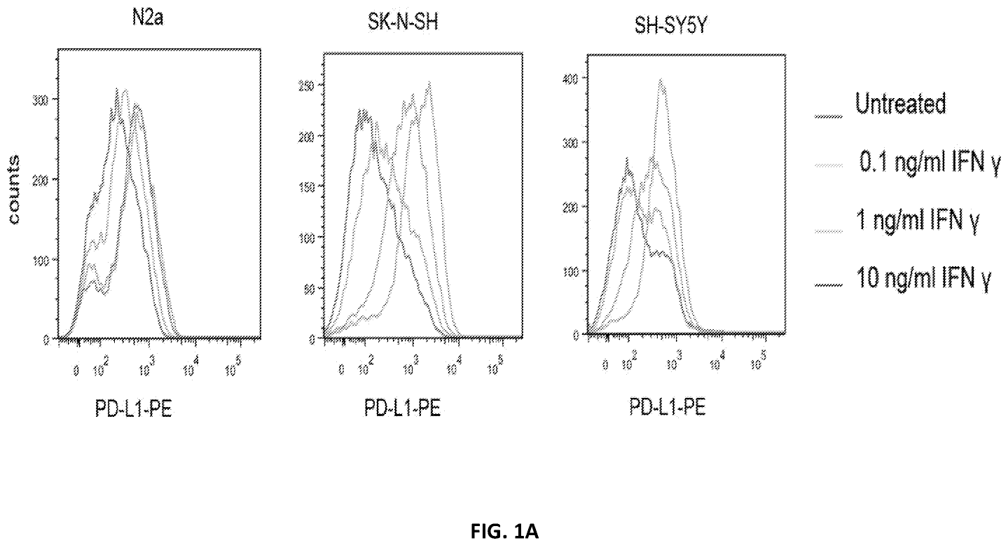

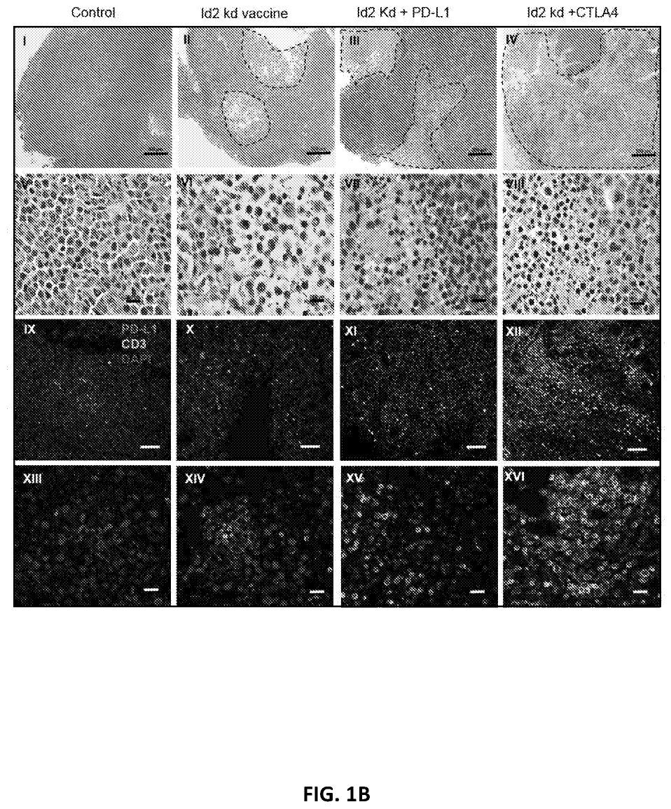

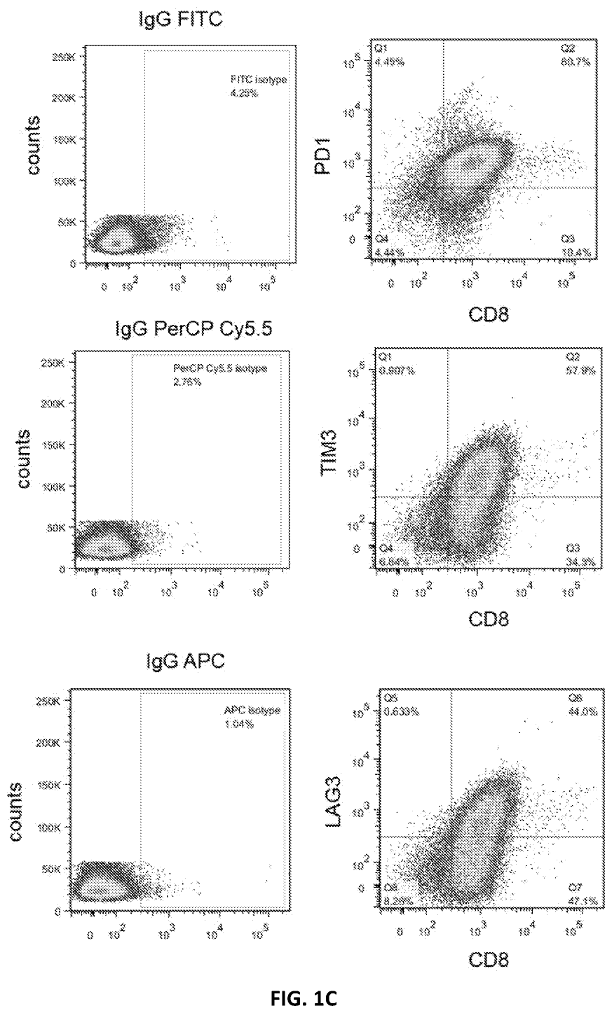

PD-L1 is detected on the mouse Neuro2a cell line, and its surface expression levels increase in a dose-dependent manner after 24 hours of stimulation with IFNγ (FIG. 1A). Similarly, the expression of PD-L1 rises markedly in response to increasing doses of IFNγ in the SK-NSH and SH-SY5Y human cell lines (non-MYCN amplified cell lines) (FIG. 1A). Thus, IFNγ produced by tumor-infiltrating T cells (TILs) may induce adaptive resistance in mouse tumors via up-regulation of PD-L1 expression.

Mouse neuroblastoma morphology and CD3+ TILs were examined following whole cell vaccination combined with CTLA-4 or PD-L1 blocking antibody. FIG. 1B, I-IV, shows that tumors from mice vaccinated with or without checkpoint inhibitors were infiltrated with leukocytes and displayed significant tumor necrosis. Tumor necrosis was most prevalent in the group that received Id2kd vaccine plus anti-CTLA-4 antibody, which also displayed the highest level of T-cell infiltrates compared to mice from the other cohorts (FIG. 1B, I-XVI). These findings indicate that the combination of immune priming (Id2kd-N2a vaccine) with immune modulation (anti-CTLA-4 antibody) potently boosts T-cell immunity. PD-L1 expression on tissues can evade immunity by binding PD1 on T cells (Freeman, et al., J. Exp. Med. (2000) 192:1027-1034; Zitvogel, et al., Oncoimmunology (2012) 1:1223-1225). To this end, it was examined whether increased T-cell infiltration induced PD-L1 tumor cell expression. A dramatic increase in PD-L1 expression was found around tumor-infiltrating lymphocytes in the mouse tumors following Id2kd plus anti-CTLA-4 treatment. Furthermore, expression levels of PD-L1 in each experimental cohort correlated with CD3⁺ T-cell influx (FIG. 1B) and was associated primarily with necrotic areas of the tumor.

CD8+ TILs were isolated from the tumors of mice treated with α-CTLA-4 plus vaccine. Flow cytometry revealed strong surface expression of PD1, TIM3, and LAG3 (FIG. 1C), which are thought to suppress cell mediated antitumor immunity. The expression of these markers may indicate the exhausted phenotype of anergic T cells, but these molecules are also reported to be activated in effector T cells (Gros, et al., (2014) J. Clin. Invest., 124:2246-2259). These findings provide the rationale that blockade of both CTLA-4 and PD-L1 will lead to improved immunotherapy by virtue of their differential targets on T-cell expansion and adaptive tumor cell resistance, respectively.

Regression, Cure, and Long-Term Immune Memory of Established Neuroblastoma Tumors with Combination Therapy

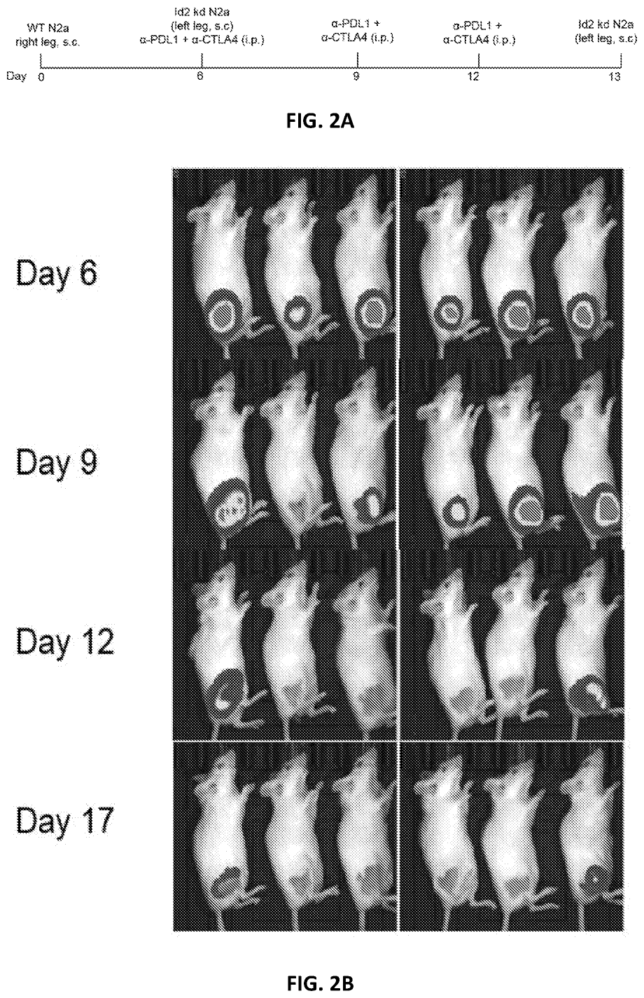

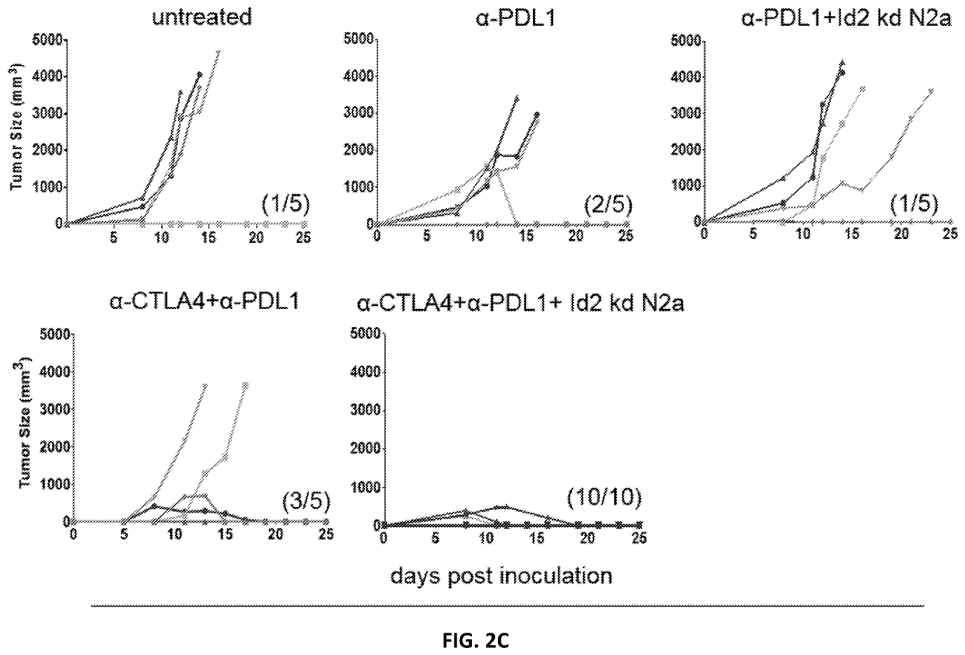

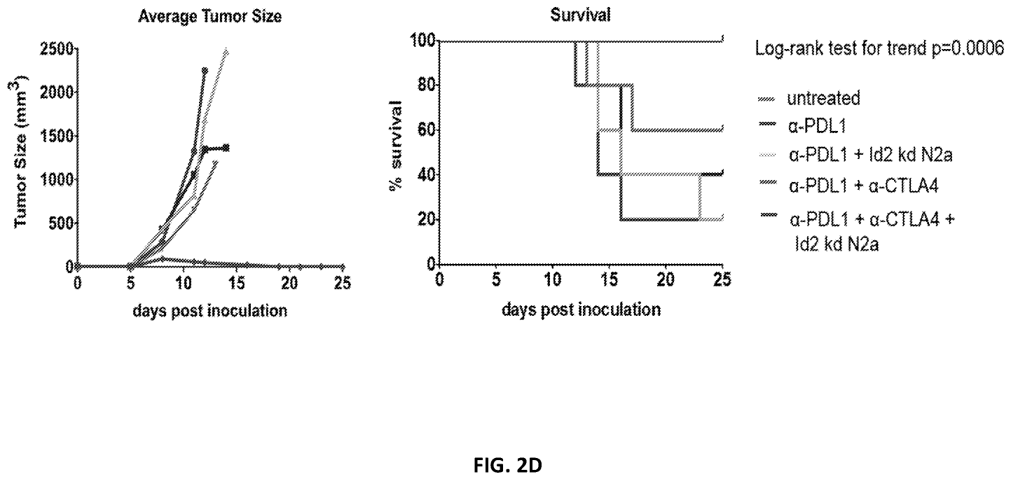

The whole cell vaccine strategy was tested in the context of both CTLA-4 and PD-L1 inhibition in a model using chemiluminescent Neuro2a cells (FIGS. 2A and 2B). Tumors were completely eradicated in all 6 mice that received the complete vaccination. In order to rule out the possibility of additional antigenicity induced by introducing chemiluminescence into the Neuro2a cells, the study was repeated using the regular Neuro2a cell line. Vaccine, anti-CTLA-4, or anti-PD-L1 alone or in combination independently with vaccination had modest effects on established tumor growth, while the combination of both anti-CTLA-4 and anti-PD-L1 without vaccination cured 60% of mice (FIG. 2C). When the vaccine was combined with both CTLA-4 and PD-L1 inhibition, all mice (n=16 in both studies) were cured of their tumors (FIGS. 2B, 2C and 2D) and remained tumor free for 6 months in follow-up (log-rank test for survival, p=0.0006) (FIG. 2D, right panel). Average tumor growth curves also showed significant differences for treatment when the combination of vaccine with anti-PD-L1 and anti-CTLA-4 was compared to control (FIG. 2D, left panel; p=0.0007, 2-way repeated measures ANOVA). These observations demonstrate the benefit of combination checkpoint therapy in which CTLA-4 inhibition expands tumor-infiltrating lymphocytes, while PD-L1 inhibition counters adaptive resistance at the tumor site.

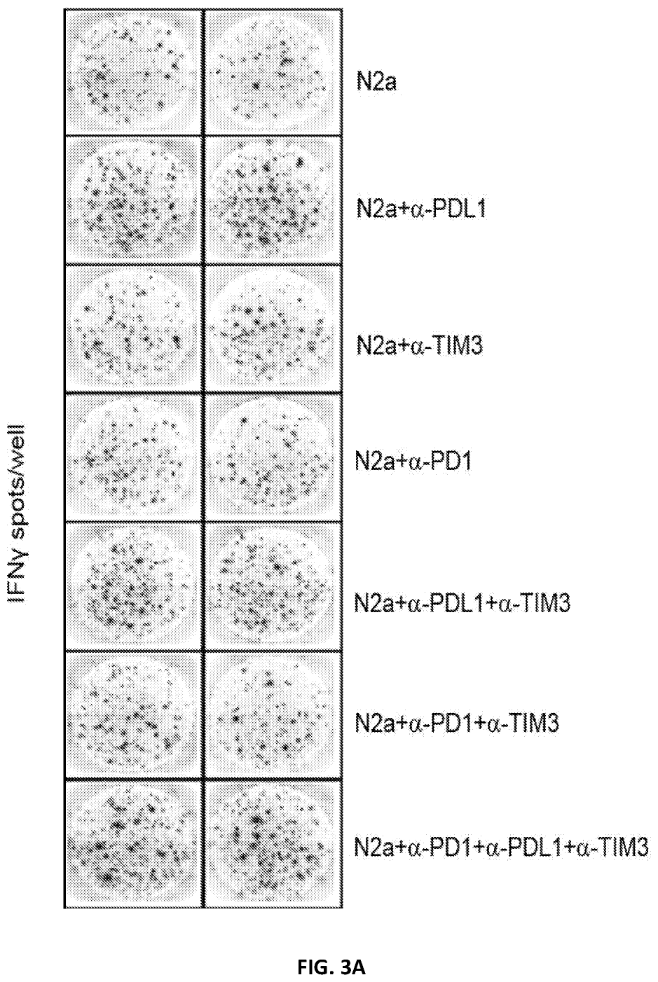

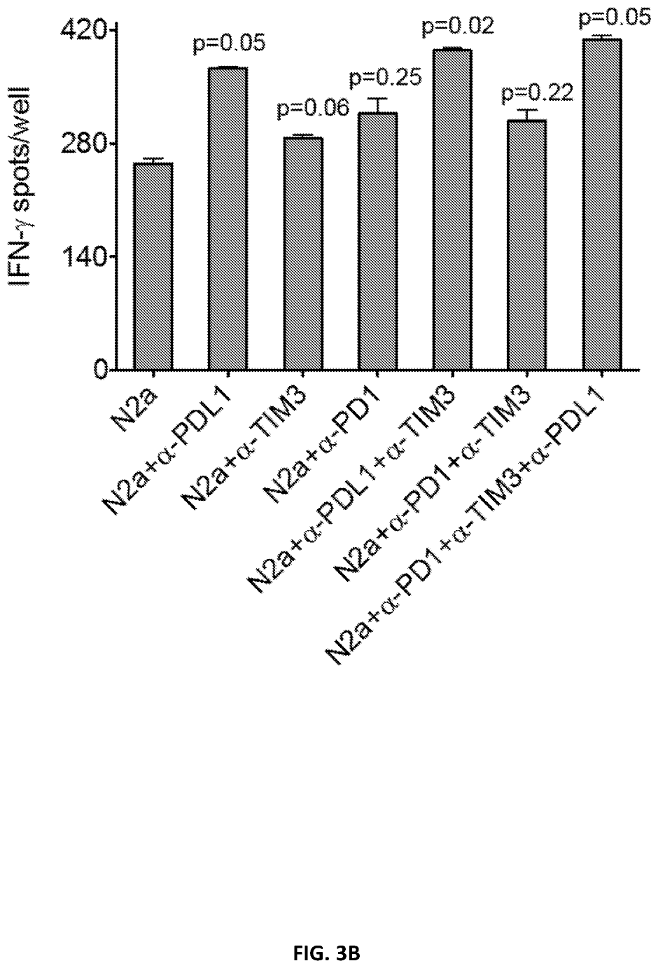

Neuro2a cells were treated with α-PD-L1 antibody to block surface expression and incubated with TILs isolated from the tumors of mice treated with α-CTLA-4 plus vaccine. Checkpoint blockers α-PD1 and α-TIM3 were also added as indicated, as these checkpoints were detected on TILs by flow cytometry. ELISpot analysis was performed, and a significant increase in IFNγ spots per well was observed only when α-PD-L1 was blocked, when compared to controls (FIG. 3A; anti-PD-L1 alone [p=0.05], anti-PD-L1 plus anti-TIM3 [p=0.02]; combined blockade with anti-PD1, PD-L1 and TIM3 [p=0.05]). In the absence of PD-L1 blocking, α-PD1 and/or anti-TIM3 did not enhance IFNγ production (unpaired 2-tailed Student t test) (FIG. 3B).

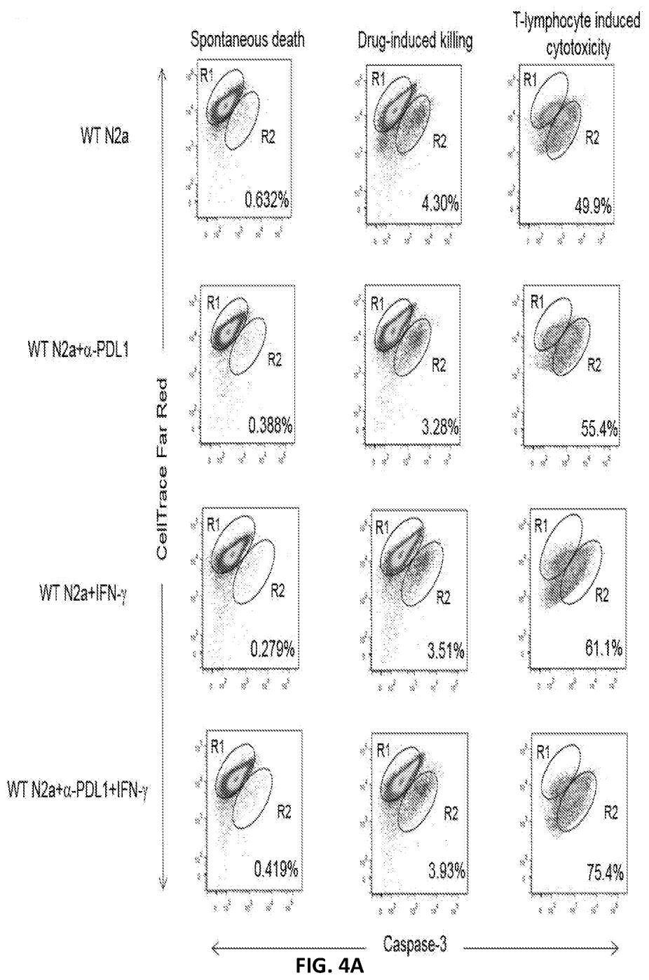

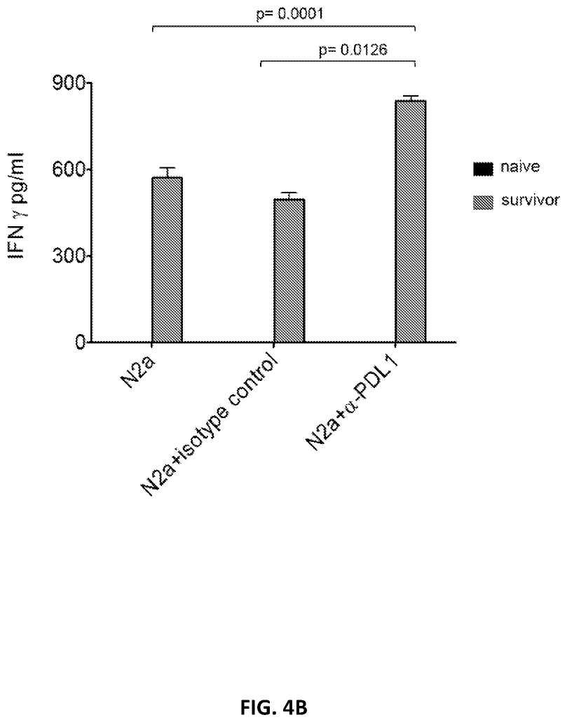

Subsequently, TILs were collected from mouse tumors at completion of the full vaccine protocol. TILs were cultured with WT Neuro2a, and a modified flow-cytometry-based cytotoxicity assay detecting the presence of activated caspase 3 in target cells was performed. Effector:target ratios of 20:1 were used for 3 hours of coculture. In WT controls, 49.3% of tumor cells underwent apoptosis, while in PD-L1 blocked targets, 73.9% of targeted tumor cells underwent apoptosis (FIG. 4A). These results confirm that effector T-cell function against neuroblastoma tumor cells is enhanced with PD-L1 blockade.

Splenocytes isolated from naïve mice and from mice that were 6-month survivors following complete vaccination with anti-PD-L1, anti-CTLA-4, and whole cell Id2kd vaccine were cocultured with Neuro2a cells in vitro. IFNγ production detected by ELISA showed potent immune memory (p=0.0126 for WT N2a cocultured with splenocytes from survivors compared with N2a blocked with α-PD-L1, and p=0.0001 when isotype control was compared to α-PD-L1, 2-tailed unpaired Student t test), whereas IFNγ responses were not detected at all in naïve splenocytes (FIG. 4B). Furthermore, survivors of vaccinated mice rechallenged with WT tumor cells rejected the challenge and failed to grow tumors. Therapeutic vaccination not only cleared established tumors but also induced long-term immune memory.

### PD-L1 Expression is Reduced in Nonimmunogenic, Aggressive Cell Lines and Tumors

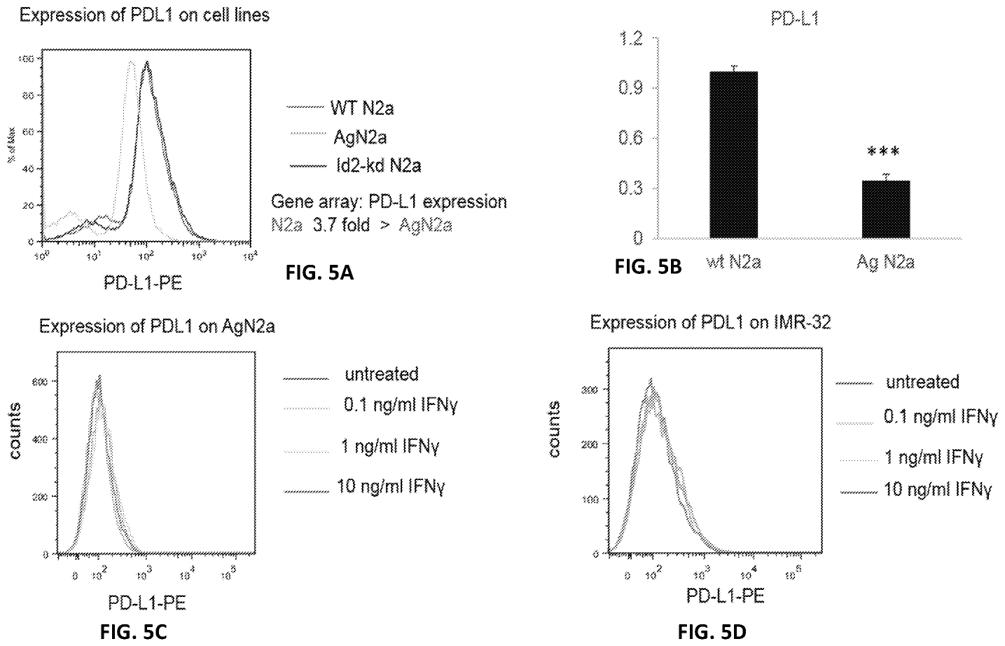

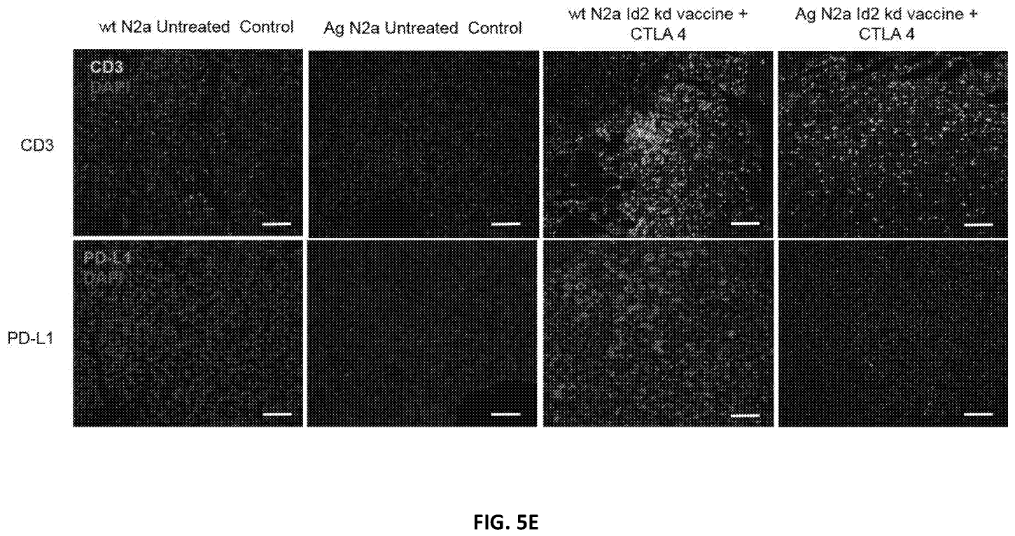

Tumor vaccination plus anti-CTLA-4 antibody was surprisingly more effective in an aggressive mouse cell line (AgN2a) (90% cure) than in the wild-type Neuro2a cell line (60% cure) (Chakrabarti, et al., PLoS ONE (2015) 10:e0129237). A possible explanation may be the differential constitutive expression of PD-L1 by tumor cells. PD-L1 expression in both mouse and human cell lines was examined, comparing mouse WT (Neuro2a) to aggressive (AgN2a) neuroblastoma and human non-MYCN-amplified SK-N-SH to MYCN-amplified IMR-32 cell lines (Schwab, et al., Proc. Natl. Acad. Sci. (1984) 81:4940-4944). MYCN amplification correlates with clinically high-risk, aggressive disease. The response of PD-L1 expression to IFNγ stimulation was also determined. Gene array data revealed a 3.5-fold-higher level of constitutive PD-L1 expression in WT N2a than in AgN2a, and this difference was verified with quantitative real-time PCR (RT-PCR) and flow cytometry (FIGS. 5A, 5B and 5C). The aggressive mouse AgN2a and the MYCN-amplified IMR-32 human cell lines failed to up-regulate PD-L1 following IFNγ treatment even at the highest concentrations tested (FIGS. 5C and 5D and FIG. 1A).

To determine whether these observations of diminished PD-L1 expression in aggressive nonimmunogenic cell lines held true for growing tumors in vivo, both the T-cell infiltrates and PD-L1 expression in AgN2a mouse tumors was examined at baseline and following vaccination. Fluorescent microscopy showed no T-cell infiltration with minimal PD-L1 expression in untreated tumors, and despite a moderate influx of CD3+ T cells following complete vaccination, the extent of induced PD-L1 expression was markedly reduced when compared to WT Neuro2a tumors sampled after vaccination (FIG. 5E).

Nonimmunogenic neuroblastoma does not up-regulate PD-L1 inhibitory pathways like immunogenic mouse neuroblastoma does. Paradoxically, this diminished adaptive resistance in aggressive nonimmunogenic tumors may enable more effective antitumor immunity.

Immunogenic Human Tumors Also Acquire PD-L1 Adaptive Resistance, which is Associated with Risk Stratification

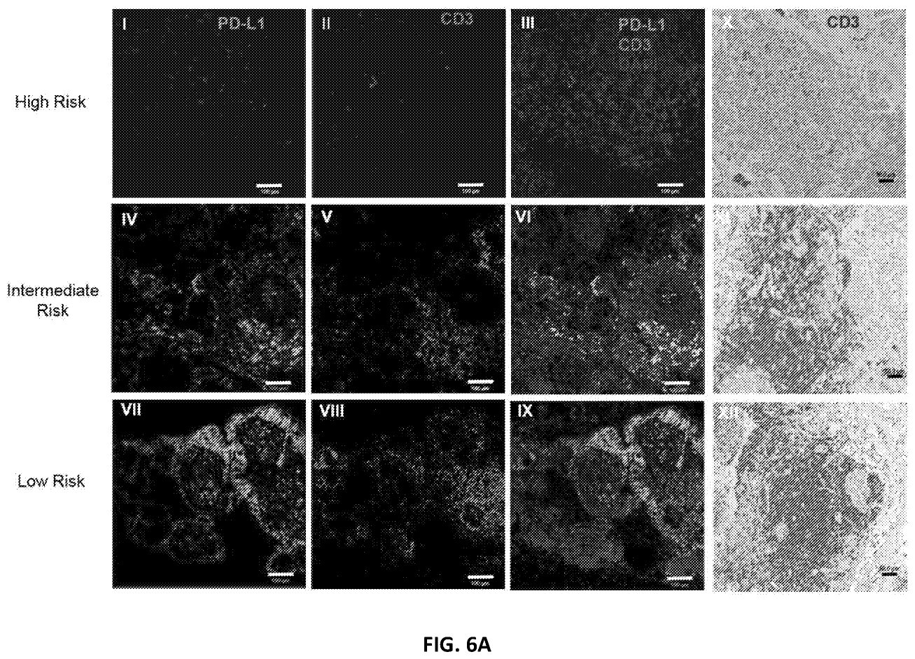

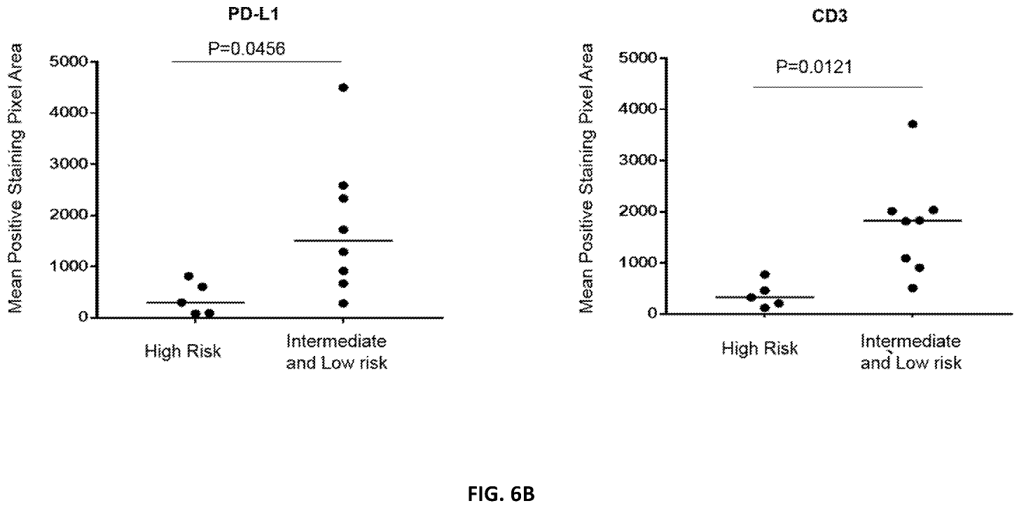

PD-L1 and CD3 expression levels in various risk-stratified tumors were cataloged from newly diagnosed and untreated human specimens (FIG. 6). The density of CD3⁺ TILs correlated with the expression of PD-L1 in human neuroblastoma tumor tissue. Dot plots showed that the distribution of CD3 and PD-L1 was statistically different between high- and intermediate/low-risk groups (FIG. 6B). In general, human NB tumors of low (n=3) and intermediate (n=5) risk had high CD3⁺ TIL cell density and marked PD-L1 expression (FIGS. 6A IV-VI and VII-IX and 6B). In contrast, high-risk (n=5) human tumors had very few CD3+ T-cell infiltrates and an absence of PD-L1 expression (FIGS. 6A I-III and 6B). These findings were similar to the mouse model in which immunogenic tumors had marked up-regulation of PD-L1 while the aggressive nonimmunogenic cell line (AgN2a) displayed minimal PD-L1 expression. These findings have implications for checkpoint immunotherapeutic strategies and also indicate that CD3⁺ TILs and PD-L1 expression are useful prognostic indicators. Since PD-L1 is induced by T-cell activity, strong PD-L1 expression in the tumor reflects an immune-suppressive microenvironment against infiltrating T cells. Only the immunogenic low/intermediate-risk tumors exploit this protective mechanism, whereas high-risk tumors are nonimmunogenic, and, thus, the PD-L1 pathway may be redundant. Taken together with the observed effects of vaccination in the mouse neuroblastoma models, PD-L1 blockade allows for effective vaccination against immunogenic tumors. High-risk nonimmunogenic tumors without TILs or PD-L1 expression will not be susceptible to checkpoint therapy alone but will be susceptible to vaccination as cell-mediated immunity can be induced against the tumor.

The work presented examines the role of adaptive immune resistance induced by PD-L1 in a mouse neuroblastoma model. Targeting PD-L1 enhanced the effectiveness of whole tumor cell vaccination, particularly when combined with CTLA-4 blockade. PD1 inhibition may be more effective than anti-CTLA-4 therapy (Parry, et al., Mol. Cell Biol. (2005) 25:9543-9553), but data presented herein indicate that this may only be true for immunogenic tumors in which tumor-infiltrating T cells are already present but rendered incompetent through inhibition of the PD1/PD-L1 pathway. The use of combination checkpoint inhibition with vaccination proved more efficacious in this mouse neuroblastoma model, which is consistent with findings in a melanoma model (Curran, et al., Proc. Natl. Acad. Sci. (2010) 107:4275-4280). The findings presented here show that CTLA-4 inhibition in the context of whole cell vaccination induced activation and expansion of TILs that were partially effective in controlling tumor growth. The TILs include both CD4+ and CD8+ subsets, but it is unclear whether CTLA-4 inhibition is acting directly on CD8 expansion or indirectly via CD4 helper function. CTLA-4 blockade may inactivate tumor-infiltrating T-reg (Spranger, et al., J. Immunother. Cancer (2014) 2:3), although work in this model did not implicate T-reg infiltration following immune cell depletion studies (Chakrabarti, et al., PLoS ONE (2015) 10:e0129237). Despite marked T-cell expansion and tumor infiltration following whole cell vaccination plus anti-CTLA-4 therapy alone, a significant proportion of tumors continued to grow (40%). The expression of PD-L1 on tumor cells induces adaptive tumor resistance. PD1 expressed on TILs is thought to be “exhausted” due to chronic stimulation by tumor antigens (Barber, et al., Nature (2006) 439:682-687), yet in the tumor model, 80% of activated TILs expressed PD1. Despite this observation, blockade of PD-L1 did not change expression of these “exhaustion” markers on the T cells themselves but rendered TILs more effective in ablating tumor growth in all mice studied and in ex vivo cellular studies. The tumor cure rate was remarkable, and the combination of checkpoint inhibition will prove critical for tumor vaccine therapy of solid tumors.

The aggressive nonimmunogenic mouse neuroblastoma (AgN2a) was surprisingly sensitive to Id2kd whole tumor cell vaccination and anti-CTLA-4 therapy alone (Chakrabarti, et al., PLoS ONE (2015) 10:e0129237). Host immunity in this model was identical to the WT immunogenic Neuro2a tumor. Thus, the tumor's lack of immune resistance may be responsible for this enhanced sensitivity. On evaluating gene array analysis of nonimmunogenic AgN2a cells compared to the parent immunogenic Neuro2a cell line, down-regulation of several tumor immunosuppressive pathways was identified, including PD-L1 (3.6-fold), CD47 (3.3-fold), CD74 (6-fold), and CD40 (2.3-fold). This finding was unexpected, but the absence of these molecular pathways speak to the lack of AgN2a tumor immunogenicity and thus redundancy for immune evasive tumor protective mechanisms. This observation indicates that nonimmunogenic tumors may be less resistant to host immunity if potent cellular immunity can be generated against the tumor. PD-L1 appears critical for generating both intrinsic and adaptive immune resistance in the wild-type Neuro2a tumor; thus, this axis was focused on in the AgN2a model. Baseline PD-L1 expression as well as IFNγ induction of PD-L1 in AgN2a was markedly reduced, as was expression in AgN2a tumors following vaccination, despite significant T-cell infiltrates. Taken together, these findings indicate that the lack of PD-L1 in AgN2a may enhance sensitivity to infiltrating TILs, which could have important implications for immunotherapy of nonimmunogenic high-risk disease. Under these conditions, the barrier to effective immune therapy in nonimmunogenic tumors would be induction of T-cell immunity. The present models indicate that whole tumor cell vaccination with Id2kd cells plus anti-CTLA-4 induces the appropriate T-cell response needed. These preclinical findings demonstrate that effective immunity can be generated against nonimmunogenic tumors and that vaccine therapy could be even more effective treatment as adaptive immune resistance seems to be of lesser significance.

Blockade of PD1/PD-L1 or CTLA-4 with other therapies has Food and Drug Administration (FDA) approval and is used against several tumor types (Barber, et al., Nature (2006) 439:682-687; Butte, et al., Immunity (2007) 27:111-122; Okazaki, et al., Nat. Immunol. (2013) 14:1212-1218; Tumeh, et al., Nature (2014) 515:568-571; Mahoney, et al., Clin. Ther. (2015) 37:764-782). Most monotherapies only achieve partial responses rather than complete responses. Combinations of checkpoint inhibitors may be more effective but are associated with more extensive adverse events when administered as nonspecific immune modulators (Wolchok, et al., N. Engl. J. Med. (2013) 369:122-133). A limitation of any immunotherapy is the potential to induce immunity against self and thus precipitate autoimmune disease or immune-related adverse events (irAEs). Therapy-induced irAEs are reported to be severe in 15%-30% of patients receiving anti-CTLA-4 alone and sometimes result in fatality (Topalian, et al., Nat. Rev. Cancer (2016) 16:275-287). Checkpoint inhibitors are frequently administered in multiple cycles until response or resistance is observed. Also, blocking PD-L1 on the target tumor could be of benefit by diminishing unwanted off-target effects, which could be less specific when blocking PD1 expression on circulating T cells.

In the present vaccine model, the combination of checkpoint inhibitors in the context of vaccine antigen, the relatively short exposure to immune modulators, and targeting inhibitory pathways on the tumor tissue itself will contribute to fewer irAEs. All surviving mice were healthy and showed no signs of irAEs when followed for at least a year, although neither specific tissue biopsies nor serum markers were followed. Despite the lack of obvious irAEs, it was also determined if a short course of vaccine therapy (6 days) against established tumor would induce significant immune memory. Mice rechallenged with tumor cells as far out as 6 months following treatment retained immune memory and rejected the tumor cell rechallenge. In support of these survival observations, marked IFNγ secretion was detected from splenocytes harvested from vaccinated mice as late as 6 months following vaccination when cultured with WT tumor cells. In the context of therapy, these findings are promising for tumor vaccination in that unlike standard therapies, immunity against the tumor is preserved and can prevent recurrence of disease following complete response with improved event-free survival (EFS).

Immunohistochemistry and confocal microscopy of mouse and human tumors allowed for imaging of the inflammatory tumor microenvironment. The relevance of immunity in the mouse neuroblastoma model to human neuroblastoma was substantiated in the study by elucidating the interplay between host response and PD-L1 in the tumor microenvironment. Low- and intermediate-risk tumors biopsied prior to any therapy had the greatest number of T-cell infiltrates. Similar to the mouse tumor findings, PD-L1 was up-regulated and associated with CD3 T-cell expression, whereas high-risk tumors had very few T cells and minimal PD-L1 expression, similar to the nonimmunogenic AgN2a aggressive tumors. High CD3 infiltrates have been noted in patients with good outcomes, while low CD3 infiltrates were associated with poor outcomes (Melaiu, et al., Clin. Cancer Res. (2017) 23(15):4462-4472). Furthermore, MYCN-amplified tumors lacked PD-L1 expression (3 of 5 high-risk tumors were MYCN amplified). However, the findings also show that both absent PD-L1 expression and high PD-L1 expression were associated with subgroups of poor-acting tumors (Melaiu, et al., Clin. Cancer Res. (2017) 23(15):4462-4472). PD-L1 expression is of particular clinical interest in that reported studies of pretreatment PD-L1 tumor expression correlated with the likelihood of anti-PD1 response in patients (Brahmer, et al., J. Clin. Oncol. (2010) 28:3167-3175; Topalian, et al., N. Engl. J. Med. (2012) 366:2443-2454). Thus, checkpoints alone in the low- and intermediate-risk inflammatory neuroblastoma tumors may be predictive of clinical response, whereas checkpoint inhibitors alone in high-risk tumors with minimal cell infiltrates will probably fail to have much predictive clinical benefit.

In conclusion, there are substantial advantages to combining checkpoint inhibitors with tumor vaccination in a model of neuroblastoma immunotherapy. Specifically, checkpoint blockade is administered in the context of tumor Ag, and thus, T-cell expansion is directed against tumor-specific antigens. CTLA-4 inhibition induces rapid proliferation and expansion of T cells, while PD-L1 blockade overcomes adaptive immune resistance on the tumor itself by enhancing the efficacy of effector T cells. More specifically, during the priming phase, CTLA-4 blockade enhances the activation and proliferation of T cells that express programmed cell death 1 (PD1) and migrate to the tumor. Programmed cell death-ligand 1 (PD-L1) is up-regulated on the tumor cells, inducing adaptive resistance. Blockade of PD-L1 allows for enhanced cytotoxic effector function of the CD8+ tumor-infiltrating lymphocytes. In the nonimmunogenic model (AgN2a), adaptive resistance through PD-L1 is of less importance. The relatively short course of immune therapy and the targeted blockade of tumor suppressive signals resulted in minimal clinical irAEs in the mouse tumor model. Despite the apparent lack of irAEs, the amplified tumor-specific immune memory is potent, protective, and of long-term duration. These critical observations are pertinent to human cancers such as neuroblastoma, for which the mouse immunogenic and nonimmunogenic neuroblastoma models mimic the inflammatory microenvironment of low/intermediate- and high-risk disease, respectively.

### Example 2

Myc is a regulator of immune escape and the knockdown of Myc in addicted cell lines induces tumor cell immunogenicity (Zhang, et al., Front. Immunol. (2017) 8:1473). Herein, it is shown that targeting Myc—MycN and c-Myc—renders tumor cells immunogenic. This observation has led to a therapeutic whole tumor cell vaccine platform that targets established tumors when combined with clinically relevant checkpoint inhibitors. Several general shortcomings of previous tumor vaccine therapy are addressed by the therapeutic whole tumor cell vaccine with checkpoint inhibitor such as: 1) facilitating presentation of multiple intact tumor antigens for antigen processing; 2) exploiting drug modified tumor cells for induction of immunogenicity; 3) countering immunosuppressive mechanisms characteristic of the tumor microenvironment and 4) targeting checkpoint inhibitors only in the context of vaccination, thereby limiting auto-immunity with long-term checkpoint therapy.

As shown in Example 1, targeting Inhibitor of differentiation protein 2 (Id2) in neuroblastoma resulted in cellular immunity when combined with checkpoint inhibition. Notably, Myc is an upstream master gene of Id proteins and effective Myc targeting suppresses Id expression as well as several other immune pathways protecting the tumor cell and enabling immune escape. Proto-oncogenes like Myc are over-expressed in most tumors and regulate proliferation, growth, differentiation and apoptosis. Myc oncogene suppresses immune surveillance and targeting Myc restores tumor immunity (Zhang, et al., Front. Immunol. (2017) 8:1473; Casey, et al., Trends Immunol. (2017) 38:298-305). By targeting Myc, the family of Id proteins (e.g., Id 1, Id2, Id3, and Id4) that are differentially expressed in various tumors can be targeted thereby expanding the utility of targeting only Id2.

In an in silico study of 148 tumors, it was determined that MYCN amplified neuroblastomas had significantly lower levels of CD45, a leukocyte marker, indicating repressed inflammatory cell infiltrates in high risk MYCN amplified neuroblastomas (Zhang, et al., Front. Immunol. (2017) 8:1473). Using the CIBERSORT algorithm (Newman, et al., Nat Methods (2015) 12(5):453-7) to estimate the percentage of each leukocyte subset, a significant reduction in all subsets (T cells, B cells, macrophages, dendritic cells and NK cells) was found in MYCN-amplified samples compared to non-MYCN amplified tumors. This result was validated by immunohistochemistry. This strong inverse correlation indicates that MYCN amplification has a profound impact on host immunity.

The potency of a whole cell vaccine combined with CTLA-4 and/or PD-L1 checkpoint blockade in a mouse neuroblastoma was demonstrated in Example 1. Indeed, a whole cell vaccine combined with CTLA-4 blockade enhanced activation and proliferation of tumor specific T-cells that express PD-1 and migrate to the tumor. PD-L1 is up-regulated on the tumor cells inducing adaptive immune resistance. Blockade of PD-L1 enhanced cytotoxic effector function of the CD8+ tumor infiltrating lymphocytes.

To determine the effect of Myc targeting on tumorigenesis, neuroblastoma N2a and melanoma B16 cells were treated in culture with several drugs that inhibit Myc transcription or target the Myc-Max interaction. Bromodomain and Extra-terminal motif (BET) proteins BRD2, BRD3, BRD4 and BRDT bind directly to acetylated lysine on histone tails to promote gene transcription by RNA polymerase II. Bromodomains are responsible for transducing the signal carried by acetylated lysine residues and translating them into various phenotypes. Bromodomain inhibitors prevent protein-protein interaction between BET proteins and acetylated histones and inhibit transcription. Highly specific inhibitors for the BET (bromodomain and extra-terminal) family of bromodomains, including I-BET726 and JQ1, can suppress oncogenes including Myc.

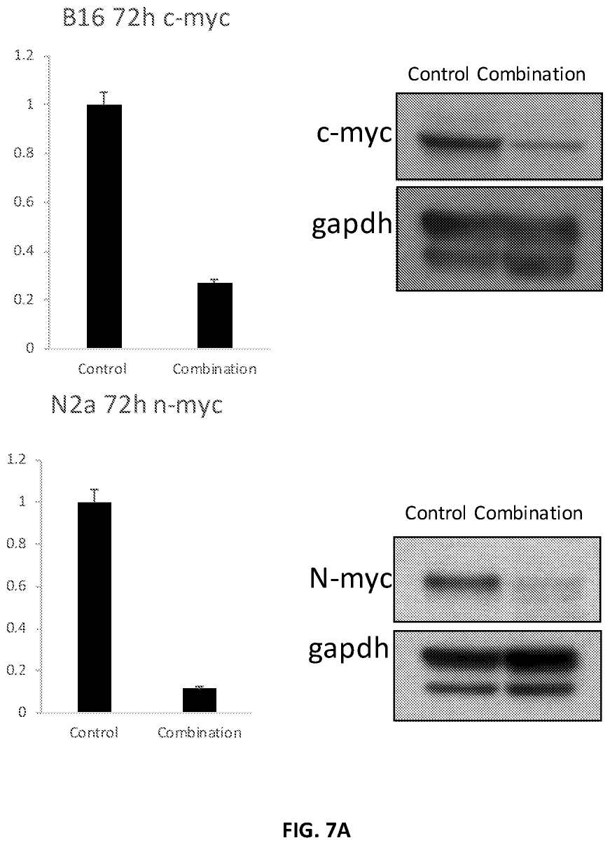

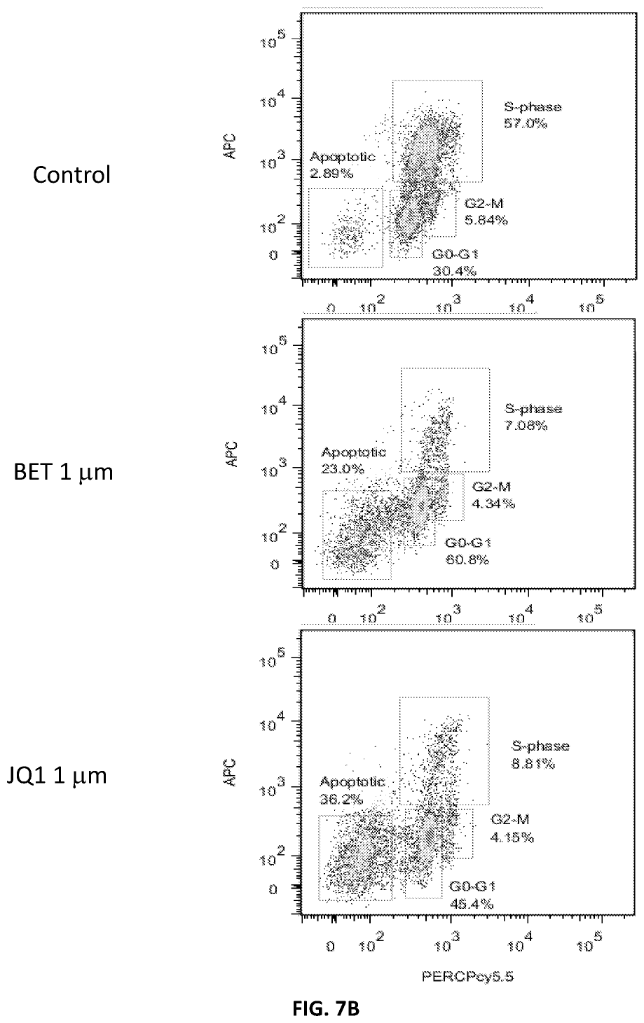

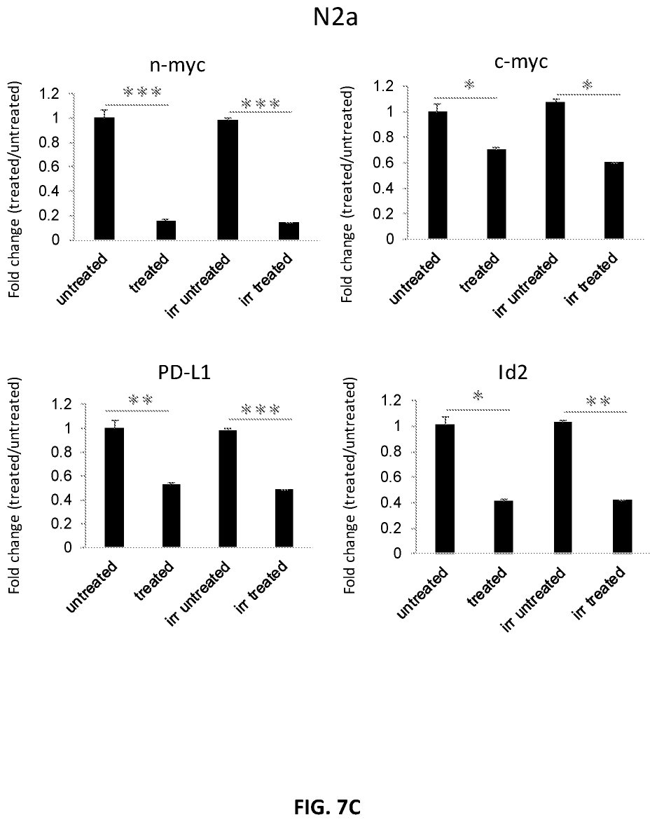

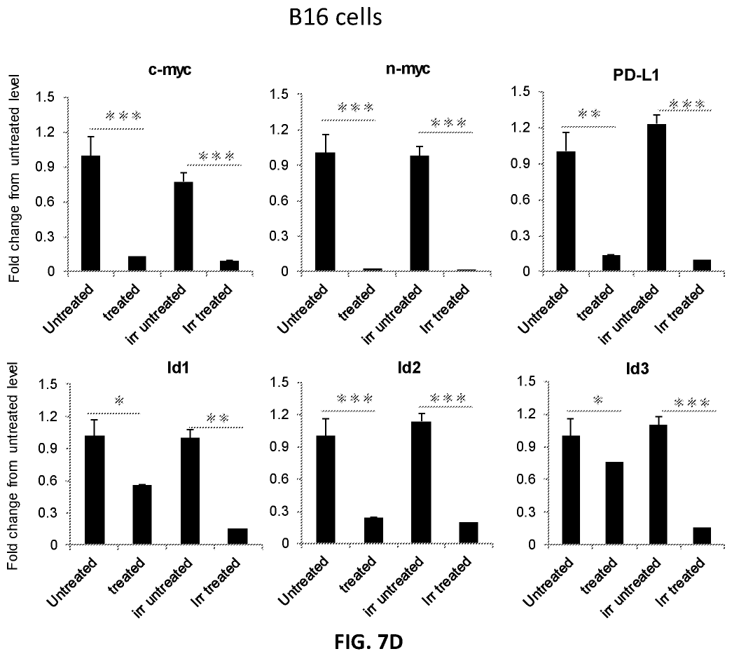

The ability of the combination of BET and JQ1 to suppressed Myc expression was tested. N2a and B16 cells were exposed to BET and JQ1 for 72 hours. Myc expression was determined by real-time PCR and Western blot analysis. As seen in FIG. 7A, both Myc N expression in the mouse neuroblastoma line (N2a) as well as c-Myc expression in the melanoma cell line (B16) were dramatically reduced. This reduction in Myc expression resulted in cell cycle arrest in the G0-G1 phase and also induced apoptosis (FIG. 7B). The cells also underwent differentiation.

N2a and B16 cells were exposed to different concentrations and/or combinations of I-BET726 and JQ1 and assessed at 3, 4, 5, 7 and 10 days. The combination of I-BET726 (0.25 μM) and JQ1 (0.25 μM) for 4 days followed by irradiation (60 Gray) was determined to completely suppress Myc genes and downstream pathways. mRNA analyses showed that myc associated genes—MycN, c-Myc, PD-L1 and Id genes—are all significantly down-regulated 2-6 fold compared with untreated cells (FIGS. 7C and 7D). Characteristically, treatment arrested the majority of cells in the G1 phase of the cell cycle and induced an arrested phenotype. However, the cells remain viable.

Treatment of cells with I-BET and JQ1 at 0.25 μM each for 4 days led to a significant reduction in cell growth relative to untreated control (control cells increased from 0.5×10⁶ to 16×10⁶, while Myc inhibitor treated cells proliferated from 0.5×10⁶ to 5×10⁶). Trypan blue staining and microscopic observation revealed that the inhibitors did not compromise cell survival. It was also apparent that there were morphological varieties in the myc-inhibited cells compared to the untreated control cells. Melanocyte differentiation coupled with an increase dendrite production. B16 cells have short dendritic processes under control culture conditions. Inhibition of myc activity resulted in a highly dendritic phenotype. Immunofluorescent staining showed nestin, which correlates with the aggressiveness and sternness of cancer cells, was significantly inhibited by Myc inhibitor in the Neuro2a cells.

Melanoma cells were also treated with I-BET/JQ1 and the expression of immune genes was studied and plotted on a heatmap. Significant up-regulation of genes associated with immunity was observed following treatment. Indeed, the genes up-regulated in treated samples were significantly higher in all pathways of inducing immunity that included antigen processing, dendritic cell function, CD molecules, MHC score, cytokine pathways, interleukin pathways, and interferon scores. Examples of upregulated cytokines included IFN-γ, IL6, TNFα, IL18, G-CSF, M-CSF CCL-5 (rantes), CXCL-1, CCL-2, CCL-7, CXCL-2, CCL3, IL-10, and IL6. The dramatic increase in the expression in immune genes was an unexpected effect of the treatment.

AP Endonuclease-1/Redox Effector Factor 1 (APE1/Ref-1) is a multifunctional enzyme involved in base excision repair (BER) that repairs oxidative base damage caused by endogenous and exogenous agents. APX3330 is an APE1/Ref-1 inhibitor and targets many of the survival pathways including HIF1a and VEGF (Logsdon, et al., Mol. Cancer Ther. (2016) 15(11):2722-2732). APX3330 (e.g., 1 μM) may be used in combination with a BET inhibitor(s) (e.g., I-BET726 and JQ1).

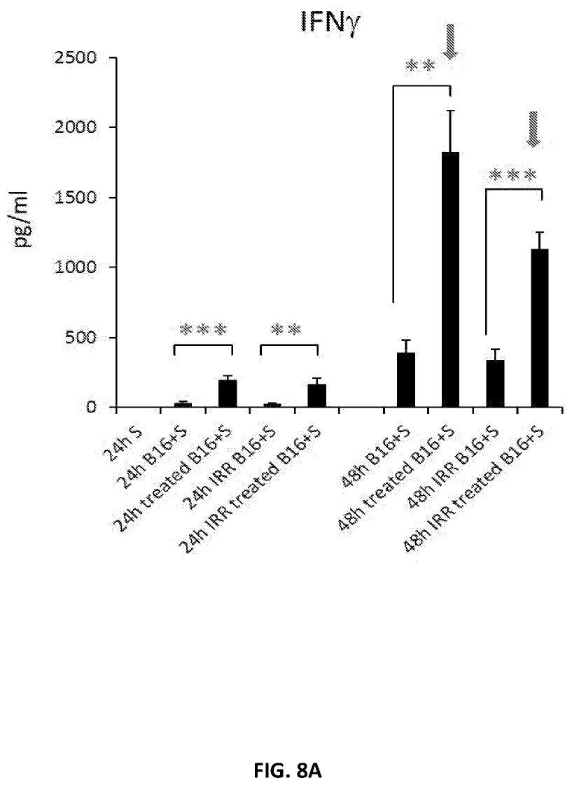

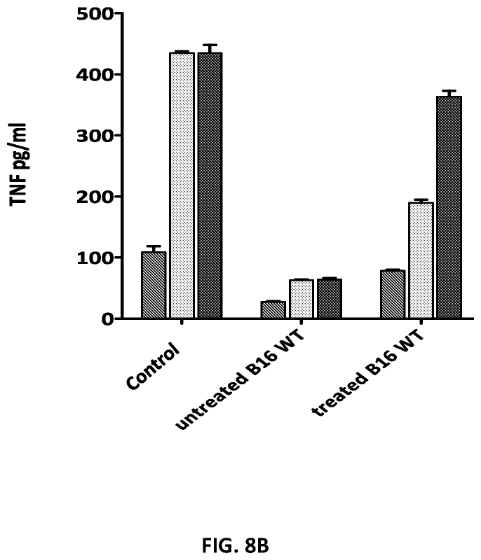

To investigate the influence of down regulation of Myc and its pathways on tumor cell immunogenicity, treated and untreated B16 cells were exposed to Myc suppressing drugs (with or without irradiation) and then co-cultured with naïve splenocytes. IFNγ production was quantified at 24 and 48 hours by ELISA. Splenocytes produced high level IFNγ only when co-cultured with Myc inhibitor treated cells (FIG. 8A). As seen in FIG. 8B, dendritic cells were inhibited by exposure to untreated wild type tumor cells but this effect was reversed when cells were pre-treated with BET/JQ1. Resiquimod (R848), which activates immune cells via TLR7/1LR8, was added to the cells. These findings indicate that down regulation of Myc in tumor cells induce tumor cell immunogenicity that stimulates host immunity. Irradiation didn't affect B16 immunogenicity.

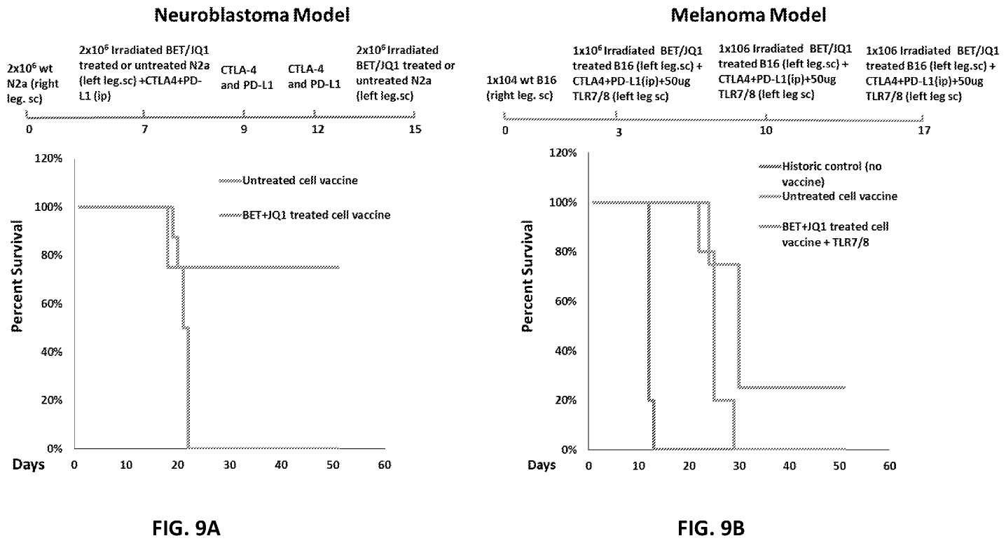

Next, the use of Myc-kd tumor cell vaccination in Neuro2a and B16 cells was tested in a therapeutic treatment model of established tumor combined with checkpoint inhibition. In the Neuro2a model, 6 of 8 mice were cured of 7 day established disease (FIG. 9A). Using the vaccine combination of Myc-kd B16 cells and checkpoint blockade in 3 day established tumors, significant delay in tumor growth was noted with 40% survival at day 30 when compared to control unvaccinated mice (FIG. 9B). The finding that knock down of the oncogene Myc renders cells immunogenic demonstrates the ability to exploit cancer cells for therapeutic tumor vaccination in the context of checkpoint inhibition.

### Example 3

In Example 2, it was shown that suppression of Myc and down-regulation of the associated molecular pathways induces tumor immunogenicity that stimulates host immunity. The novel therapeutic vaccine was determined to be more effective in the neuroblastoma tumor model than in the melanoma model. To evaluate immune differences in cell lines, a NanoString™ Mouse PanCancer Immune Profiling Panel analysis was performed that profiles 752 immune related genes. One of the most interesting differences between the Neuro2a (neuroblastoma) and B16 (melanoma) cell lines was the level of ApoE, which was >2000 fold higher in the melanoma cell line than the neuroblastoma cell line. ApoE can inhibit both antigen presenting cell (APC) function as well as T-cell function. For example, ApoE can suppress lymphocyte proliferation and generate cytolytic T-cells. Based on the functions of ApoE, it was postulated that ApoE could be protecting tumor cells that express it by inhibiting immune cell function. Thus, it was determined whether targeting ApoE in the context of tumor vaccination would enhance efficacy as a novel target for dis-inhibiting T-cell function.

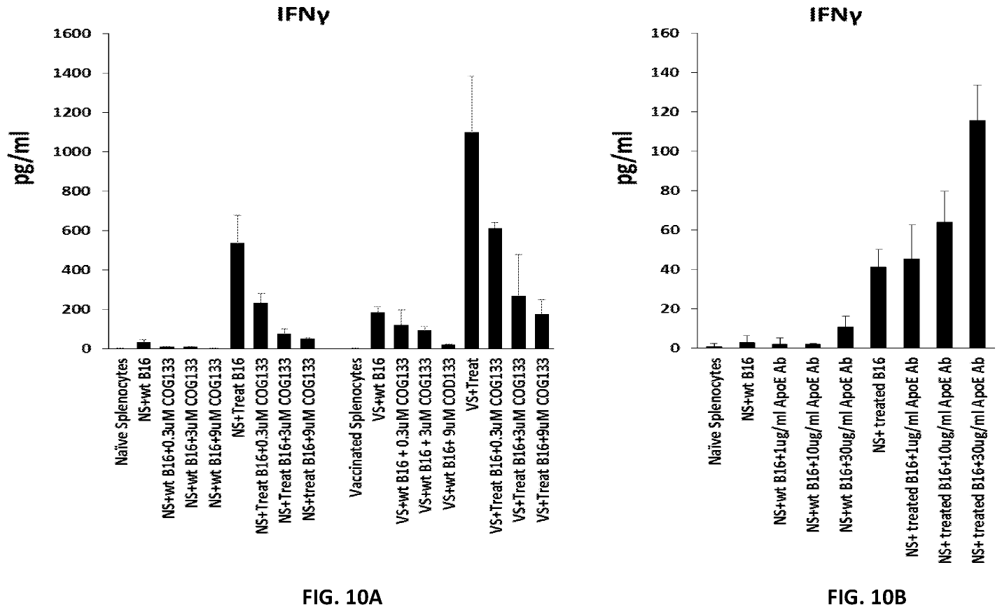

B16 cells were exposed to Myc suppressing drugs (0.25 μM BET+0.25 μM JQ1) for 4 days, and then irradiated at 60 Gy and subsequently co-cultured with vaccinated splenocytes in the presence of either ApoE agonist peptide fragment COG133 (LRVRLASHLRKLRKRLL (SEQ ID NO: 1); 0.3, 3 and 9 μM) or anti-ApoE antibody (1, 10 and 30 μg/ml). IFNγ production was quantified by ELISA at 48 hours. Results showed that splenocytes produced high levels of IFNγ only when co-cultured with Myc inhibitor treated cells (FIGS. 10A and 10B). In addition, exposure of these cells to ApoE agonist COG133 repressed IFNγ production (6 fold reduction) (FIG. 10A) as well as TNFα and Rantes, while the presence of anti-ApoE antibody induced enhanced IFNγ production (3 fold increase) (FIG. 10B). These results show that ApoE is a negative regulator of activated T cell function. These finding also explain the discrepancy in vaccine efficacy between Neuro2a and B16. The vaccine is very effective in the Neuro2a model with a >80% cure, while in the B16 model despite prolonging survival, the cure rate was lower at −20%. However, B16 has >2000 fold increase in ApoE levels in the cell line and may suppress activated T-cell function. These observations provide evidence of ApoE's role in suppressing immunity.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating cancer in a subject in need thereof, said method comprising:
A) contacting cancer cells obtained from the subject to be treated with an inhibitor of an immunity suppressing tumor protein;
B) rendering said cancer cells proliferation-incompetent; and
C) administering said cancer cells and a checkpoint inhibitor to the subject, thereby treating said cancer.

2. The method of claim 1, wherein said inhibitor of an immunity suppressing tumor protein is an inhibitor of Myc, Inhibitor of differentiation protein 2 (Id2), and/or apolipoprotein E (ApoE).

3. The method of claim 2, wherein said inhibitor of an immunity suppressing tumor protein is an inhibitor of Myc.

4. The method of claim 3, wherein the Myc inhibitor is an inhibitor of Bromodomain and Extra-terminal motif (BET) proteins.

5. The method of claim 4, wherein the BET inhibitor is JQ1 or I-BET726.

6. The method of claim 1, wherein step A) comprises contacting the cancer cells with JQ1 and I-BET726.

7. The method of claim 3, wherein the Myc inhibitor is 10058-F4.

8. The method of claim 1, wherein said checkpoint inhibitor is selected from the group consisting of programmed cell death (PD-1) inhibitors, programmed cell death-ligand 1 (PD-L1) inhibitors, and CTLA-4 inhibitors.

9. The method of claim 8, wherein said checkpoint inhibitor is an antibody.

10. The method of claim 9, wherein step C) comprises administering an anti-PD-L1 antibody and an anti-CTLA-4 antibody.

11. The method of claim 1, further comprising administering an inhibitor of ApoE.

12. The method of claim 1, further comprising obtaining a biological sample from the subject and isolating said cancer cells prior to step A).

13. The method of claim 1, wherein step B) comprises irradiating said cancer cells.

14. A method of stimulating an immune response to a tumor in a subject in need thereof, said method comprising:
A) contacting cancer cells obtained from the tumor in the subject with an inhibitor of an immunity suppressing tumor protein;
B) rendering said cancer cells proliferation-incompetent; and
C) administering said cancer cells and a checkpoint inhibitor to the subject, thereby stimulating an immune response to said tumor.

15. A method of producing a whole cell tumor vaccine, said method comprising:
A) contacting cancer cells obtained from a subject having a cancer or tumor with an inhibitor of an immunity suppressing tumor protein; and
B) rendering said cancer cells proliferation-incompetent, thereby generating said whole cell tumor vaccine.

16. The method of claim 15, further comprising contacting said cancer cells with a checkpoint inhibitor.

17. The method of claim 15, further comprising contacting said cancer cells with an inhibitor of ApoE.

18. The method of claim 15, wherein said inhibitor of an immunity suppressing tumor protein is an inhibitor of Myc.

19. The method of claim 18, wherein the Myc inhibitor is an inhibitor of Bromodomain and Extra-terminal motif (BET) proteins.

20. The method of claim 15, wherein step A) comprises contacting the cancer cells with JQ1 and I-BET726.

21. A method of treating cancer in a subject in need thereof, said method comprising administering a whole cell tumor vaccine and a checkpoint inhibitor to the subject, thereby treating said cancer.

22. The method of claim 21, wherein said checkpoint inhibitor is selected from the group consisting of programmed cell death (PD-1) inhibitors, programmed cell death-ligand 1 (PD-L1) inhibitors, and CTLA-4 inhibitors.

23. The method of claim 22, wherein said checkpoint inhibitor is an antibody.

24. The method of claim 22, comprising administering an anti-PD-L1 antibody and an anti-CTLA-4 antibody to the subject.


SEQUENCE LISTING (1 sequence)

SEQ ID NO: 1 - protein, 17 aa, Artificial Sequence
  COG133
LRVRLASHLRKLRKRLL